SEQUENCE LISTING (18 sequences)

SEQ ID NO: 1 - RNA, 8461 nt, Artificial Sequence
  hopK0-RBD
augggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcuguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagaauggccaccaugagaagaaugcagcugcugcugcugaucgcccugagccuggcccuggugacaaacagcagggugcagccuaccgagagcaucgugagauuucccaacaucaccaaucugugccccuucggcgagguguuuaaugccaccagauucgccagcguguacgccuggaauaggaagaggaucagcaacugcguggccgacuacuccgugcuguacaauuccgccuccuuuuccacauucaaguguuacggcguguccccuaccaagcugaacgaucuguguuucaccaauguguacgccgacagcuucgugaucaggggcgaugaggugaggcagaucgccccuggccagacaggcaagaucgccgacuacaacuacaagcugcccgaugacuuuacaggcugugugaucgccuggaacuccaacaaccuggacagcaaggugggcggcaauuacaauuaccuguacagacuguucagaaagagcaaccugaagccuuucgagagggacaucuccaccgagaucuaccaggccggcuccacacccuguaauggcguggagggcuucaacuguuacuucccccugcagagcuacggcuuucagcccaccaacggcgugggcuaccagccuuacagggugguggugcugagcuuugagcugcugcacgccccugccacagugugcggcccaaagaaguccaccaaccuggugaagaauaagugugugaauuucuaacaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaauaggg

SEQ ID NO: 2 - RNA, 8908 nt, Artificial Sequence
  hopK1-RBD
augggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcuguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagaauggccaccaugagaagaaugcagcugcugcugcugaucgcccugagccuggcccuggugacaaacagcagggugcagccuaccgagagcaucgugagauuucccaacaucaccaaucugugccccuucggcgagguguuuaaugccaccagauucgccagcguguacgccuggaauaggaagaggaucagcaacugcguggccgacuacuccgugcuguacaauuccgccuccuuuuccacauucaaguguuacggcguguccccuaccaagcugaacgaucuguguuucaccaauguguacgccgacagcuucgugaucaggggcgaugaggugaggcagaucgccccuggccagacaggcaagaucgccgacuacaacuacaagcugcccgaugacuuuacaggcugugugaucgccuggaacuccaacaaccuggacagcaaggugggcggcaauuacaauuaccuguacagacuguucagaaagagcaaccugaagccuuucgagagggacaucuccaccgagaucuaccaggccggcuccacacccuguaauggcguggagggcuucaacuguuacuucccccugcagagcuacggcuuucagcccaccaacggcgugggcuaccagccuuacagggugguggugcugagcuuugagcugcugcacgccccugccacagugugcggcccaaagaaguccaccaaccuggugaagaauaagugugugaauuucaugcagaagggcgaccagaauccucagaucgccgcccacgugaucuccgaggccagcagcaagacaacauccgugcugcagugggccgagaagggcuacuacaccaugagcaacaaccuggugacacuggagaacggcaagcagcugaccgugaagaggcagggccuguacuacaucuacgcccaggugacauuuugcagcaauagagaggccuccagccaggcccccuucaucgccagccugugucugaaguccccuggcagauuugagagaauccugcugagggccgccaacacccacagcagcgccaagccuugcggccagcaguccauccaccugggcggcguguuugagcugcagcccggcgcuuccguguuugugaacgugaccgauccuagccaggugagccacggcaccggcuuuacauccuucggccugcugaagcuguaacaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaauaggg

SEQ ID NO: 3 - RNA, 44 nt, Artificial Sequence
  hopK1-RBD SAM 5′UTR
augggcggcgcaugagagaagcccagaccaauuaccuacccaaa

SEQ ID NO: 4 - RNA, 7482 nt, Artificial Sequence
  hopK1-RBD SAM nonstru replicase
auggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcuguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaa

SEQ ID NO: 5 - RNA, 50 nt, Artificial Sequence
  K1-RBD SAM 5′nonstru
ccugaauggacuacgacauagucuaguccgccaagucuagaauggccacc

SEQ ID NO: 6 - RNA, 729 nt, Artificial Sequence
  hopK1-RBD SAM GOI
augagaagaaugcagcugcugcugcugaucgcccugagccuggcccuggugacaaacagcagggugcagccuaccgagagcaucgugagauuucccaacaucaccaaucugugccccuucggcgagguguuuaaugccaccagauucgccagcguguacgccuggaauaggaagaggaucagcaacugcguggccgacuacuccgugcuguacaauuccgccuccuuuuccacauucaaguguuacggcguguccccuaccaagcugaacgaucuguguuucaccaauguguacgccgacagcuucgugaucaggggcgaugaggugaggcagaucgccccuggccagacaggcaagaucgccgacuacaacuacaagcugcccgaugacuuuacaggcugugugaucgccuggaacuccaacaaccuggacagcaaggugggcggcaauuacaauuaccuguacagacuguucagaaagagcaaccugaagccuuucgagagggacaucuccaccgagaucuaccaggccggcuccacacccuguaauggcguggagggcuucaacuguuacuucccccugcagagcuacggcuuucagcccaccaacggcgugggcuaccagccuuacagggugguggugcugagcuuugagcugcugcacgccccugccacagugugcggcccaaagaaguccaccaaccuggugaagaauaagugugugaauuuc

SEQ ID NO: 7 - RNA, 450 nt, Artificial Sequence
  hopK1-RBD SAM CD40L
augcagaagggcgaccagaauccucagaucgccgcccacgugaucuccgaggccagcagcaagacaacauccgugcugcagugggccgagaagggcuacuacaccaugagcaacaaccuggugacacuggagaacggcaagcagcugaccgugaagaggcagggccuguacuacaucuacgcccaggugacauuuugcagcaauagagaggccuccagccaggcccccuucaucgccagccugugucugaaguccccuggcagauuugagagaauccugcugagggccgccaacacccacagcagcgccaagccuugcggccagcaguccauccaccugggcggcguguuugagcugcagcccggcgcuuccguguuugugaacgugaccgauccuagccaggugagccacggcaccggcuuuacauccuucggccugcugaagcuguaa

SEQ ID NO: 8 - RNA, 108 nt, Artificial Sequence
  hopK1-RBD SAM 3′ UTR
caauuggcaagcugcuuacauagaacucgcggcgauuggcaugccgccuuaaaauuuuuauuuuauuuuucuuuucuuuuccgaaucggauuuuguuuuuaauauuuc

SEQ ID NO: 9 - RNA, 45 nt, Artificial Sequence
  hopK1-RBD SAM POLY A
aaaaaaaaaaaaaaaaaaaaaaaaaaaaaaaaaaaaaaaauaggg

SEQ ID NO: 10 - RNA, 11555 nt, Artificial Sequence
  S1+S2 SAM
augggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcuggccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauucuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagcauauggccaccauguucguguuucuggugcugcugccucugguguccagccagugugugaaccugaccaccagaacacagcugccuccagccuacaccaacagcuuuaccagaggcguguacuaccccgacaagguguucagauccagcgugcugcacucuacccaggaccuguuccugccuuucuucagcaacgugaccugguuccacgccauccacguguccggcaccaauggcaccaagagauucgacaaccccgugcugcccuucaacgacgggguguacuuugccagcaccgagaaguccaacaucaucagaggcuggaucuucggcaccacacuggacagcaagacccagagccugcugaucgugaacaacgccaccaacguggucaucaaagugugcgaguuccaguucugcaacgaccccuuccugggcgucuacuaccacaagaacaacaagagcuggauggaaagcgaguuccggguguacagcagcgccaacaacugcaccuucgaguacgugucccagccuuuccugauggaccuggaaggcaagcagggcaacuucaagaaccugcgcgaguucguguucaagaacaucgacggcuacuucaagaucuacagcaagcacaccccuaucaaccucgugcgggaucugccucagggcuucucugcucuggaaccccugguggaucugcccaucggcaucaacaucacccgguuucagacacugcuggcccugcacagaagcuaccugacaccuggcgauagcagcagcggauggacagcuggugccgccgcuuacuaugugggcuaccugcagccuagaaccuuccugcugaaguacaacgagaacggcaccaucaccgacgccguggauugugcccuugauccucugagcgagacaaagugcacccugaaguccuucaccguggaaaagggcaucuaccagaccagcaacuuccgggugcagcccaccgaauccaucgugcgguuccccaauaucaccaaucugugccccuucggcgagguguucaaugccaccagauucgccucuguguacgccuggaaccggaagcggaucagcaauugcguggccgacuacuccgugcuguacaacuccgccagcuucagcaccuucaagugcuacggcguguccccuaccaagcugaacgaccugugcuucacaaacguguacgccgacagcuucgugauccggggagaugaagugcggcagauugccccuggacagacaggcaagaucgccgacuacaacuacaagcugcccgacgacuucaccggcugugugauugccuggaacagcaacaaccuggacuccaaagucggcggcaacuacaauuaccuguaccggcuguuccggaaguccaaucugaagcccuucgagcgggacaucuccaccgagaucuaucaggccggcagcaccccuuguaacggcguggaaggcuucaacugcuacuucccacugcaguccuacggcuuucagcccacaaauggcgugggcuaucagcccuacagagugguggugcugagcuucgaacugcugcaugccccugccacagugugcggcccuaagaaaagcaccaaucucgugaagaacaaaugcgugaacuucaacuucaacggccugaccggcacaggcgugcugacagagagcaacaagaaguuccugccauuccagcaguuuggccgggauaucgccgauaccacagacgccguuagagauccccagacacuggaaauccuggacaucaccccuugcagcuucggcggagugucugugaucaccccuggcaccaacaccagcaaucagguggcagugcuguaccaggacgugaacuguaccgaagugcccguggccauucacgccgaucagcugacaccuacauggcggguguacuccaccggcagcaauguguuucagaccagagccggcugucugaucggagccgagcacgugaacaauagcuacgagugcgacauccccaucggcgcuggcaucugugccagcuaccagacacagacaaacagccccagacgggccagaucuguggccagccagagcaucauugccuacacaaugucucugggcgccgagaacagcguggccuacuccaacaacucuaucgcuauccccaccaacuucaccaucagcgugaccacagagauccugccuguguccaugaccaagaccagcguggacugcaccauguacaucugcggcgauuccaccgagugcuccaaccugcugcugcaguacggcagcuucugcacccagcugaauagagcccugacagggaucgccguggaacaggacaagaacacccaagagguguucgcccaagugaagcagaucuacaagaccccuccuaucaaggacuucggcggcuucaauuucagccagauucugcccgauccuagcaagcccagcaagcggagcuucaucgaggaccugcuguucaacaaagugacacuggccgacgccggcuucaucaagcaguauggcgauugucugggcgacauugccgccagggaucugauuugcgcccagaaguuuaacggacugacagugcugccuccucugcugaccgaugagaugaucgcccaguacacaucugcccugcuggccggcacaaucacaagcggcuggacauuuggagcuggcgccgcucugcagauccccuuugcuaugcagauggccuacagauucaacggcaucggagugacccagaaugugcuguacgagaaccagaagcugaucgccaaccaguucaacagcgccaucggcaagauccaggacagccugagcagcacagcaagcgcccugggaaagcugcaggacguggucaaccagaaugcccaggcacugaacacccuggucaagcagcuguccuccaacuucggcgccaucagcucugugcugaacgauauccugagcagacuggacccuccugaggccgaggugcagaucgacagacugaucacaggcagacugcagagccuccagacauacgugacccagcagcugaucagagccgccgagauuagagccucugccaaucuggccgccaccaagaugucugagugugugcugggccagagcaagagaguggacuuuugcggcaagggcuaccaccugaugagcuucccucagucugccccucacggcgugguguuucugcacgugacauacguucccgcucaagagaagaauuucaccaccgcuccagccaucugccacgacggcaaagcccacuuuccuagagaaggcguguucguguccaacggcacccauugguucgugacacagcggaacuucuacgagccccagaucaucaccaccgacaacaccuucgugucuggcaacugcgacgucgugaucggcauugugaacaauaccguguacgacccucugcagcccgagcuggacagcuucaaagaggaacuggacaaguacuuuaagaaccacacaagccccgacguggaccugggcgauaucagcggaaucaaugccagcgucgugaacauccagaaagagaucgaccggcugaacgagguggccaagaaucugaacgagagccugaucgaccugcaagaacuggggaaguacgagcaguacaucaaguggcccugguacaucuggcugggcuuuaucgccggacugauugccaucgugauggucacaaucaugcuguguugcaugaccagcugcuguagcugccugaagggcuguuguagcuguggcagcugcugcaaguucgacgaggacgauucugagcccgugcugaagggcgugaaacugcacuacacaugagcggccgcgaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaacgcg

SEQ ID NO: 11 - RNA, 8694 nt, Artificial Sequence
  popV0-RBD SAM
augggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcuguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagcgccaccaugcgcagaaugcagcugcugcugcugaucgcccugagccuggcccuggugaccaacagcucuagaagagugcagcccaccgagagcaucgugcgcuuccccaacaucaccaaccugugccccuucggcgagguguucaacgccacccgguucgccuccguguacgccuggaaccggaagcgcaucagcaacugcguggccgacuacuccgugcuguacaacuccgccuccuucuccaccuucaagugcuacggcgugagccccaccaagcugaacgaccugugcuucaccaacguguacgccgacuccuucgugauccggggcgacgaggugcggcagaucgccccuggacagaccggcaagaucgccgacuacaacuacaagcugcccgacgacuucaccggcugcgugaucgccuggaacuccaacaaccuggacuccaaggugggcggcaacuacaacuaccuguaccggcuguuccggaaguccaaccugaagcccuucgagcgggacaucagcaccgagaucuaccaggccggcagcacccccugcaacggcguggaaggcuucaacugcuacuucccccugcagagcuacggcuuccagcccaccaacggcgugggcuaccagcccuaccgcgugguggugcuguccuucgagcugcugcacgcccccgccaccguguguggaccuaagaaguccaccaaccuggugaagaacaagugcgugaacuucuaaggcgcgccuauguuacgugcaaaggugauugucaccccccgaaagaccauauugugacacacccucaguaucacgcccaaacauuuacagccgcggugucaaaaaccgcguggacgugguuaacaucccugcugggaggaucagccguaauuauuauaauuggcuuggugcuggcuacuauuguggccauguacgugcugaccaaccagaaacauaauugaauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaauaggg

SEQ ID NO: 12 - RNA, 9147 nt, Artificial Sequence
  popV1-RBD SAM
augggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcuguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagcgccaccaugcgcagaaugcagcugcugcugcugaucgcccugagccuggcccuggugaccaacagcucuagaagagugcagcccaccgagagcaucgugcgcuuccccaacaucaccaaccugugccccuucggcgagguguucaacgccacccgguucgccuccguguacgccuggaaccggaagcgcaucagcaacugcguggccgacuacuccgugcuguacaacuccgccuccuucuccaccuucaagugcuacggcgugagccccaccaagcugaacgaccugugcuucaccaacguguacgccgacuccuucgugauccggggcgacgaggugcggcagaucgccccuggacagaccggcaagaucgccgacuacaacuacaagcugcccgacgacuucaccggcugcgugaucgccuggaacuccaacaaccuggacuccaaggugggcggcaacuacaacuaccuguaccggcuguuccggaaguccaaccugaagcccuucgagcgggacaucagcaccgagaucuaccaggccggcagcacccccugcaacggcguggaaggcuucaacugcuacuucccccugcagagcuacggcuuccagcccaccaacggcgugggcuaccagcccuaccgcgugguggugcuguccuucgagcugcugcacgcccccgccaccguguguggaccuaagaaguccaccaaccuggugaagaacaagugcgugaacuucacgcguaugcacaagggcgaccaggacccccagaucgccgcccaugugaucagcgaggccuccagcaagaccgccuccgugcugcagugggcccccaagggauacuacacccugagcaccaaccuggugacccuggagaacggcagacagcuggccgugaagcgccagggcaucuacuacaucuacgcccaggugaccuucugcuccaaccgggacgccgccggccaggcuccuuucaucgccucccugugccugcggagccccuccggauccgagagaauccugcugcgcgccgccaacacccacuccuccagcaagcccugcggccagcaguccauccaccugggcggcguguucgagcugcagcccggagcuuccguguucgugaacgugaccgaccccucccaggugucccacggcaccggauucaccuccuucggccugcugaagcuguaaggcgcgccuauguuacgugcaaaggugauugucaccccccgaaagaccauauugugacacacccucaguaucacgcccaaacauuuacagccgcggugucaaaaaccgcguggacgugguuaacaucccugcugggaggaucagccguaauuauuauaauuggcuuggugcuggcuacuauuguggccauguacgugcugaccaaccagaaacauaauugaauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaauaggg

SEQ ID NO: 13 - RNA, 44 nt, Artificial Sequence; identical to SEQ ID NO: 3
  popV1-RBD SAM 5′ UTR

SEQ ID NO: 14 - RNA, 7482 nt, Artificial Sequence; identical to SEQ ID NO: 4
  popV1-RBD SAM non-struc replicase

SEQ ID NO: 15 - RNA, 735 nt, Artificial Sequence
  popV1-RBD SAM GOI
augcgcagaaugcagcugcugcugcugaucgcccugagccuggcccuggugaccaacagcucuagaagagugcagcccaccgagagcaucgugcgcuuccccaacaucaccaaccugugccccuucggcgagguguucaacgccacccgguucgccuccguguacgccuggaaccggaagcgcaucagcaacugcguggccgacuacuccgugcuguacaacuccgccuccuucuccaccuucaagugcuacggcgugagccccaccaagcugaacgaccugugcuucaccaacguguacgccgacuccuucgugauccggggcgacgaggugcggcagaucgccccuggacagaccggcaagaucgccgacuacaacuacaagcugcccgacgacuucaccggcugcgugaucgccuggaacuccaacaaccuggacuccaaggugggcggcaacuacaacuaccuguaccggcuguuccggaaguccaaccugaagcccuucgagcgggacaucagcaccgagaucuaccaggccggcagcacccccugcaacggcguggaaggcuucaacugcuacuucccccugcagagcuacggcuuccagcccaccaacggcgugggcuaccagcccuaccgcgugguggugcuguccuucgagcugcugcacgcccccgccaccguguguggaccuaagaaguccaccaaccuggugaagaacaagugcgugaacuuc

SEQ ID NO: 16 - RNA, 53 nt, Artificial Sequence
  popV1-RBD SAM 5′ non-stru GOI
ccugaauggacuacgacauagucuaguccgccaagucuagcgccaccacgcgu

SEQ ID NO: 17 - RNA, 450 nt, Artificial Sequence
  popV1-RBD SAM CD40L
augcacaagggcgaccaggacccccagaucgccgcccaugugaucagcgaggccuccagcaagaccgccuccgugcugcagugggcccccaagggauacuacacccugagcaccaaccuggugacccuggagaacggcagacagcuggccgugaagcgccagggcaucuacuacaucuacgcccaggugaccuucugcuccaaccgggacgccgccggccaggcuccuuucaucgccucccugugccugcggagccccuccggauccgagagaauccugcugcgcgccgccaacacccacuccuccagcaagcccugcggccagcaguccauccaccugggcggcguguucgagcugcagcccggagcuuccguguucgugaacgugaccgaccccucccaggugucccacggcaccggauucaccuccuucggccugcugaagcuguaa

SEQ ID NO: 18 - RNA, 338 nt, Artificial Sequence
  popV1-RBD SAM 3′ UTR
ggcgcgccuauguuacgugcaaaggugauugucaccccccgaaagaccauauugugacacacccucaguaucacgcccaaacauuuacagccgcggugucaaaaaccgcguggacgugguuaacaucccugcugggaggaucagccguaauuauuauaauuggcuuggugcuggcuacuauuguggccauguacgugcugaccaaccagaaacauaauugaauacagcagcaauuggcaagcugcuuacauagaacucgcggcgauuggcaugccgccuuaaaauuuuuauuuuauuuuucuuuucuuuuccgaaucggauuuuguuuuuaauauuuc

(19) United States
(12) Patent Application Publication
(10) Pub. No.: US 2022/0395571 A1
(43) Pub. Date: Dec. 15, 2022

(54) mRNA VACCINE AND METHOD OF INDUCING ANTIGEN-SPECIFIC IMMUNE RESPONSES IN INDIVIDUALS

(71) Applicant: Abnova (Taiwan) Corporation, Taipei (TW)

(72) Inventor: WILBER HUANG, Taipei (TW)

(73) Assignee: Abnova (Taiwan) Corporation, Taipei (TW)

(21) Appl. No.: 17/836,990

(22) Filed: Jun. 9, 2022

(30) Foreign Application Priority Data
Jun. 11, 2021 (TW) 110121479

(51) Int. Cl. A61K 39/215; A61P 31/14; C12N 7/00; A61P 11/00; A61K 39/00

(52) U.S. Cl. CPC A61K 39/215; A61P 31/14; C12N 7/00; A61P 11/00; A61K 2039/53

(57) ABSTRACT

An mRNA vaccine includes one or more polynucleotides and a pharmaceutically acceptable vector. Each polynucleotide includes a coding region. The coding region includes a gene of interest and a ligand sequence which encodes a CD40 ligand.

### SEQUENCE LISTING

The instant application contains a Sequence Listing which has been submitted electronically in ASCII format and is hereby incorporated by reference in its entirety. Said ASCII copy, created on Jul. 14, 2022, is named ABN-P0019-USA_ST25.txt and is 87,096 bytes in size.

### BACKGROUND OF THE INVENTION

### 1. Field of the Invention

The present invention generally relates to a messenger RNA vaccine, and a method of inducing an antigen-specific immune response in an individual. In particular, the present invention is directed to a messenger RNA vaccine which is able to induce a specific antigen, and a method to use a messenger RNA vaccine which can produce a specific antigen in the presence of a ligand sequence to induce an antigen-specific immune response in an individual.

### 2. Description of the Prior Art

A vaccine is one of the most important medicines for the treatment of or prevention of various diseases. For example, a vaccine may be a prophylactic or therapeutic vaccine. A vaccine is a biological composition to actively acquire immunity after the induction against a specific disease. Traditionally, vaccines have been designed to contain agents which resemble pathogenic microorganisms. The agents are formulated to stimulate a human body's immune system to produce antibodies or cytokines in advance such that the antibodies or cytokines which are associated with the agents can recognize and inhibit any microorganisms which may be encountered in the future.

The designs of most commercial vaccines or vaccines in development are based on the whole microorganisms, protein antigens, peptides, polysaccharides, or the combinations thereof. For example, deoxyribonucleic acid (DNA) vaccine is a technique which is used to stimulate and generate cellular immune responses. However, the DNA used in this technique may be integrated into an individual's genome to lead to potential risks, including possible insertional mutagenesis, which may result in the activation of oncogenes or the inhibition of tumor suppressor genes.

In addition, RNA vaccines, such as messenger RNA vaccines, may be used to treat and/or prevent a variety of diseases. The design of RNA vaccines is based on intervening of RNA in the translational process of cellular metabolism to assign individual cells to produce appropriate translation products, such as protein antigens, or peptides. However, the design and efficacy of existing RNA vaccines still have problems for applications. For example, the titers of cellular immune response are still insufficient.

Therefore, there is still a lack of RNA vaccines which are easy to design, simple to manufacture, and can exhibit sufficient neutralizing antibody titers in cellular immune responses. Also, there is a lack of a method to induce sufficient antigen-specific immune responses in individuals to cope with the public health crisis of an increasingly severe global pandemic.

### SUMMARY OF THE INVENTION

In view of these, the present invention proposes a messenger RNA vaccine, and a method which is able to induce a sufficient antigen-specific immune response in an individual. The messenger RNA vaccine which is proposed by the present invention is not only easy to design, simple to manufacture, but also shows sufficient neutralizing antibody titers in the immune response. In addition, after the administration of the RNA vaccines proposed in the present invention, sufficient antigen-specific immune responses may also be induced in individuals to be beneficial to cope with the public health crisis of an increasingly severe global pandemic.

In one aspect, the present invention first proposes a messenger RNA vaccine. The messenger RNA vaccine proposed by the present invention may include one or more polynucleotides and a pharmaceutically acceptable vector. Each one of the one or more polynucleotides may include a 5′ end cap region, an untranslated region (UTR), a coding region, and a polyadenosine tail region (poly-A). The coding region may include a gene of interest (GOI), and a ligand sequence which encodes a CD40 ligand. A pharmaceutically acceptable vector may be used to encapsulate one or more polynucleotides.

In an embodiment of the present invention, the coding region may further include a functional sequence which encodes a viral replicase.

In another embodiment of the present invention, the functional sequence may be a self-amplifying functional sequence, so that the messenger RNA vaccine may be accordingly optimized to be a self-amplifying messenger RNA (SAM) vaccine.

In another embodiment of the present invention, the gene of interest may include a sequence of interest encoding a receptor-binding domain (RBD).

In another embodiment of the present invention, the receptor-binding domain may be the receptor-binding domain of the spike protein of a severe acute respiratory syndrome coronavirus (SARS-CoV).

In another embodiment of the present invention, the gene of interest may include to encode at least one of the S1 subunit and the S2 subunit of a spike protein of a severe acute respiratory syndrome coronavirus 2 (SARS-CoV-2).

In another embodiment of the present invention, the pharmaceutically acceptable vector may be selected from the group consisting of lipid nanoparticles (LNP) and polymeric nanoparticles (PNP).

In another aspect, the present invention proposes a method of inducing an antigen-specific immune response in an individual. The method of the present invention may include the administration of a messenger RNA vaccine in an amount effective to generate an antigen-specific immune response in an individual. The messenger RNA vaccine may include a ligand sequence which encodes a CD40 ligand.

In one embodiment of the invention, the antigen-specific immune response may include a T cell response.

In another embodiment of the invention, the antigen-specific immune response may include a B cell response.

In another embodiment of the present invention, the method of the present invention may include the administration of one or more doses of the messenger RNA vaccine.

In another embodiment of the invention, the messenger RNA vaccine may be administered to an individual by subcutaneous injection or by intramuscular injection.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 shows the analysis data of the absorbance at 450 nm using the self-amplifying messenger RNA vaccine of the present invention by enzyme-linked immunosorbent assay (ELISA).

FIG. 2 shows comparison data of the serum IC₅₀ titers between conventional messenger RNA vaccines and the messenger RNA vaccines of the present invention.

FIG. 3A, FIG. 3B, FIG. 3C, FIG. 3D, FIG. 3E, FIG. 3F respectively show the neutralizing ability correlation data of the collected mouse sera for IgG immune responses to the receptor-binding domain 35 days and 42 days after the administration of the messenger RNA vaccines of the present invention.

FIG. 4A, FIG. 4B, FIG. 4C, FIG. 4D, FIG. 4E, and FIG. 4F respectively show the neutralizing ability data of the collected mouse sera for IgG immune responses to the receptor-binding domain 35 days and 42 days after the administration of the messenger RNA vaccines of the present invention.

FIG. 5A, FIG. 5B, FIG. 5C, FIG. 5D, FIG. 5E, and FIG. 5F respectively show the neutralizing ability data of the collected mouse sera for IgG immune responses to the receptor-binding domain 35 days and 42 days after the administration of the messenger RNA vaccines of the present invention encapsulated in different vectors.

FIG. 6A shows the neutralizing ability data of the collected mouse sera for IgG immune responses to the receptor-binding domain 49 days after the administration of the messenger RNA vaccines of the present invention.

FIG. 6B shows show the neutralizing ability data of the collected mouse sera for IgG immune responses to the receptor-binding domain 182 days after the administration of the messenger RNA vaccines of the present invention.

### DETAILED DESCRIPTION

Unless otherwise specified, all technical and scientific terms used herein have the meanings commonly understood by one of ordinary skill in the art. Terms used herein in the specification are for the purpose of describing particular embodiments only and are not construed to limit the scope of the present invention. Unless stated to the contrary, terms used in the specification and in the claims have the following meanings.

Antigen: As used herein, an antigen is a protein or a peptide which induces an immune response, and may be encoded by the administered messenger ribonucleic acid. Typically, an antigen is a foreign substance which induces an immune response. In this case, the antigen may substantially be a foreign protein, such as a spike protein, but may also be a non-protein. Without being limited for the purposes of the present invention, an antigen means the antigen from protein or from a polypeptide which is encoded by the administered messenger ribonucleic acid to induce or is expected to induce an immune response.

As used herein, the term deliver may include local, topical and systemic delivery. For example, the delivery of messenger RNAs may include the delivery of the messenger RNAs to a target tissue, and may further include the expression of the encoded protein within the target tissue.

As used herein, the term “encapsulation or encapsulate” may refer to the state of accommodation or of confinement of one or more messenger RNA molecules within a carrier or within a vector.

As used herein, expression of a nucleic acid sequence may refer to translate messenger RNA in a cell into polypeptides, to assemble multiple polypeptides into complete proteins (for example, enzymes), and/or post-translational modifications of polypeptides or fully assembled proteins (for example, enzymes). In the present invention, the terms “express” and “produce” or the like may be used interchangeably.

As used herein, an improvement, an increase, or a decrease, or similar terms, may denote an amount of change of a measured value relative to a baseline value.

As used herein, the term in vitro refers to an event that occurs in an artificial environment, such as in a test tube, in a reaction vessel or in cell culture . . . etc., rather than in an organism.

As used herein, the term “in vivo” refers to an event that occurs within an organism, such as within a cell in humans and in non-human animals. For the description regarding a cell-related event, it may refer to an event which occurs within living cells (as opposed to for example in vitro systems).

As used herein, the term messenger ribonucleic acid (mRNA) may refer to a polynucleotide encoding one or more polypeptides. As used herein, a messenger ribonucleic acid may include both a modified and an unmodified ribonucleic acid. A single messenger ribonucleic acid may include one or more coding regions or one or more non-coding regions. A messenger ribonucleic acid may be purified from a natural source, produced or optionally purified using a recombinant expression system, or chemically synthesized, and the like. A messenger ribonucleic acid sequence is presented in a direction from 5′ to 3′ orientation unless otherwise indicated.

As used herein, a mutant virus or a variant virus may refer to a virus whose amino acid sequence differs from a wild or a reference sequence. For example, a coronavirus is a ribonucleic acid (RNA) virus, which is easy to mutate during replication. It is currently known that SARS-CoV-2 has a variety of mutant strains, such as B.1.1.7 mutant, B.1.351 mutant or B.1.617 mutant. The spike protein (S protein) of these mutant strains all include more or less variations in the amino acid sequence, resulting in the amino acid sequence variants different from those of the wild strain. The mutation of the virus is conducive to the immune escape of the virus. Compared to the wild or reference sequence, the variations in the amino acid sequence may include substitutions, deletions and/or insertions at specific positions within the amino acid sequence. In some embodiments, the amino acid sequence of variations may be at least 80% identical to a wild or a reference sequence.

As used herein, when referring to a polypeptide or a polynucleotide, the term feature may be defined as the components of the molecules based on different amino acid sequences or based on nucleotides, respectively. Features of polypeptides encoded by polynucleotides include surface appearances, local conformational shapes, folds, loops, half-loops, domains, half-domains, sites, ends, or any combination thereof.

As used herein, when referring to a polypeptide, the term binding domain may refer to a motif of a polypeptide having one or more identifiable structural or functional features or properties (for example, binding capacity, serving as a site for protein-protein interactions).

As used herein, a messenger RNA vaccine may be used as a therapeutic or a prophylactic agent. It may be used in a medicine to prevent and/or treat an infectious disease.

As used herein, antibody titers may be a measure of the correspondingly produced antibodies in an individual, for example, antibodies specific for a particular antigen (for example, the spike protein of a coronavirus) or an epitope of an antigen. Antibody titers are usually expressed as the reciprocal of the largest dilution that gives a positive result. An enzyme-linked immunosorbent assay (ELISA) is a commonly used assay for the determination of antibody titers, for example.

As used herein, the term subject or individual may refer to any organism to which a provided vaccine is administered, for example, for experimental, diagnostic, prophylactic, cosmetic, and/or therapeutic purposes. A typical individual may include an animal, for example, mammals, such as mice, rats, rabbits, non-human primates, and/or humans. In some embodiments, the individual may be a human.

As used herein, the term “pharmaceutically acceptable” may refer to a substance suitable for use in contact with human tissues or with animal tissues without undue toxicity, irritation, allergic reaction or other problems or complications to be commensurate with a reasonable benefit/risk ratio within the scope of sound medical judgment.

As used herein, the terms “subcutaneous administration or subcutaneous injection” may refer to the administration to a subcutaneous tissue, which is the layer of the tissue between the skin and muscle.

As used herein, the term effective amount may refer to an amount sufficient to prevent and/or delay the onset of symptoms when the amount is administered to an individual. As will be understood by those of ordinary skill in the art, effective dosage may generally be administered by a dosage regimen including at least one unit dose.

As used herein, the term vaccination may refer to a composition to generate an immune response, such as by the administration of a messenger RNA-encoded antigen. For the purposes of the present invention, the vaccination may be performed before, during and/or after exposure to a causative factor, in some embodiments prior to exposure to the factor. In some embodiments, vaccination may include multiple administrations of a vaccine composition, possibly appropriately spaced in time.

A messenger RNA vaccine is a novel vaccine and offers many advantages over existing cell-based vaccines, for example, using live, attenuated or pathogen-killed or toxoid vaccines. In addition to safety, a messenger RNA vaccine is cost-effective and offers a flexible design platform. A messenger RNA encoding specific antigens may be involved in inducing specific immune responses so it may be used to develop therapeutic or prophylactic messenger RNA vaccines against a variety of diseases.

Unexpectedly, according to various embodiments of the present invention, the inventors of the present invention developed a class of formulations for the delivery of a messenger RNA in vivo for use as a vaccine. The messenger RNA vaccine of the present invention, such as, but not limited to, the messenger RNA vaccine of SEQ ID NO: 1, SEQ ID NO: 2, SEQ ID NO: 10, SEQ ID NO: 11 or SEQ ID NO: 12, may have significantly enhanced or synergistic immune responses in many aspects, including the production of functional antibodies with sufficient neutralizing ability along with enhanced titers. These results are possibly achieved even when dosages no greater than those used for other classes of vaccines are administered. The messenger RNA vaccines of the present invention may exhibit neutralizing antibodies of sufficient titers in cellular immune responses, and may have prophylactic efficacy.

In addition, a self-amplifying messenger RNA vaccine may exhibit an excellent function of the self-amplifying messenger RNA by its included coding sequence corresponding to a viral replicase. The messenger RNA vaccine of the present invention may produce enough protein by itself to induce a sufficiently strong immune response. Therefore, in some embodiments, the self-amplifying messenger RNA vaccine of the present invention may have the function of self-replicating messenger RNA, and may include non-structural protein coding regions required for the virus replication, such as, but not limited to the sequences of, SEQ ID NO:4, SEQ ID NO:5, SEQ ID NO:14, or SEQ ID NO:16.

In some embodiments, the messenger RNA vaccine of the present invention may use lipid nanoparticles (LNP) or polymer nanoparticles as a vector to encapsulate one or more polynucleotides, to significantly enhance the effectiveness of the messenger RNA vaccine, or to significantly enhance the in vivo efficacy of the vaccine of the present invention.

According to the experimental data in some embodiments of the present invention, compared with existing vaccines, it is confirmed that messenger RNA vaccines may produce stronger neutralizing antibody titers, and the cellular immune response caused by the ligand sequences within may be much higher than that caused by a protein antigen. In mice, the messenger RNA vaccines of the present invention may induce a balanced and stable immune response of IgG antibodies in which the first-type helper T cells (Th1) and the second-type helper T cells (Th2) participate together, and can achieve significant neutralizing antibody titers. For example, according to the results of some experiments, it is known that the amount of IgG antibodies induced by the messenger RNA vaccines of the present invention is sufficient for a prophylactic method or for a therapeutic method.

In some embodiments, the present invention may provide a method of inducing an antigen-specific immune response in an individual. For example, the method for inducing an antigen-specific immune response in an individual of the present invention may include the administration of an amount of a messenger RNA vaccine effective to generate an antigen-specific immune response to the individual. For example, an amount of the messenger RNA vaccine to effectively generate an antigen-specific immune response may include one or more polynucleotides, for example but not limited to, SEQ ID NO:1, SEQ ID NO:2, SEQ ID NO: 10, SEQ ID NO:11 or SEQ ID NO:12, of messenger RNAs or of sequences. One of the polynucleotides may include one or more coding regions. A coding region may include a gene of interest and a ligand sequence encoding a CD40 ligand. In some embodiments, the present invention further provides a method of delivering a messenger RNA encoding a target protein to an individual to induce an antigen-specific immune response. An exemplary, non-limiting sequence abstract of a messenger RNA of the present invention may be shown as follows.

5′ end cap region-5′ untranslated region-coding region (gene of interest+CD40 ligand sequence)-3′ untranslated region-3′ poly-adenosine tail region (3′-poly-A tail region)

In some embodiments, polynucleotides may be used as messenger RNAs of the present invention. A messenger RNA may refer to any encoded polynucleotide which encodes one or more naturally occurring, non-naturally occurring or modified amino acid polymers and may be translated in vitro or in vivo to produce one or more polypeptides.

By encapsulating the messenger RNAs in a carrier, designed messenger RNAs may be efficiently delivered to induce an antigen-specific immune response so that the antigens which can induce an antigen-specific immune response, for example but not limited to the spike protein of severe acute respiratory syndrome-related coronavirus, may be expressed in vivo. Therefore, the messenger RNAs which encode antigenic peptides or protein may be used to produce the same in vivo efficacy of vaccination to have the industrial utility. The messenger RNA vaccines may be administered to an individual one or more times to generate sufficient antibodies, for example, may be administered one, two, three, four, or more than four times. The way of administering one or more times of messenger RNA vaccines may be, for example, by subcutaneous injection or by intramuscular injection, but the present invention is not limited thereto. The exact amount required for the present methods of administering a messenger RNA vaccine may vary among individuals depending on the species, the age, general conditions of the individual, the severity of the disease, the particular compositions, the modes of administration, the modes of activity, and the like.

For example, mammalian individuals can translate the components in the vaccine to produce the corresponding encoded antigenic peptides or proteins, thereby generating a corresponding immune response, to further protect the host from subsequent infections by similar pathogens. This process is called preventive vaccination. In addition, a vaccine may also strengthen the host immune system against a current infection, for example, by redirecting the immune response against new microbial antigens and then inducing a stronger immune response, thereby eliminating the pathogen's route of infection. Vaccines of this type of immune response mechanism can be classified as inducing an antigen-specific immune response in an individual. These antigen-specific immune responses may include at least one of T cell responses, B cell responses, or helper T cell responses. During the process of these immune responses, T cells, B cells, or helper T cells may be activated, resulting in the subsequent T cell memory and the humoral immune response.

In one aspect, the present invention first proposes a messenger RNA vaccine. The messenger RNA vaccine of the present invention may be a composition or a formulation. The coding regions in the messenger RNA vaccine of the present invention may be combined or the components of the messenger RNA vaccine of the present invention may be formulated to generate an effective amount of antibodies specific to an antigen-specific immune response in an individual. The messenger RNA vaccines of the present invention can produce an effective amount of specific antibodies, for example but not limited to IgG antibodies, in an individual.

An effective amount of IgG antibodies produced by the messenger RNA vaccines of the present invention may be, for example but not limited to, one or more IgG antibodies. For example, T cells can be further divided into Th1 subtypes and Th2 subtypes according to the cytokines which they secrete. For example, the Th2 subtype can correspond to IgG1 antibodies and the Th1 subtype corresponds to IgG2a antibodies. The effective amount of IgG antibodies produced by the messenger RNA vaccines of the present invention may include, for example IgG1 antibodies and IgG2a antibodies, but the present invention is not limited thereto.

For example, the experimental results show that: a variety of messenger RNA vaccines of the present invention 1. a dosage of 5 μg, with the gene of interest to be S1+S2 of the spike protein and PNP as the vector; 2. a dosage of 5 μg, with human codon optimization, with anti-kanamycin, with the gene of interest to be the receptor-binding domain, with CD40 ligand and LNP as the vector; 3. a dosage of 5 μg, with the gene of interest to be S1+S2 of the spike protein and LNP as the vector, can produce a sufficient amount of IgG1 antibodies corresponding to the Th2 subtype, and IgG2a antibodies corresponding to the Th1 subgroup. In some embodiments, the produced ratio of (IgG1 antibody/IgG2a antibody) can be close to 1, which means that the messenger RNA vaccines of the present invention can separately and sufficiently activate Th1 subtype-related and Th2 subtype-related immunity reactions.

The messenger RNA vaccines of the present invention may include one or more polynucleotides, and a pharmaceutically acceptable vector. In some embodiments, the basic composition of an effective messenger RNA molecule can generally include a 5′-end cap region, a 5′ untranslated region (5′ UTR), at least one coding region, a 3′ untranslated region (3′ UTR), and a 3′ poly-A tail region (poly-A). The polynucleotides provided by the present invention may be used as messenger RNAs. However, the messenger RNAs of the present invention can be distinguished from wild-type or conventional messenger RNA molecules in terms of their functional and/or structural design features, and these features may render the messenger RNAs of the present invention show inventive step and industrial utility.

In some embodiments, the messenger RNA vaccines of the present invention may include one or more polynucleotides. One of the polynucleotides may have an open reading frame encoding at least one viral antigenic protein. The messenger RNAs of the present invention may be encapsulated in a vector, such as lipid nanoparticles to facilitate the drug delivery to target cells, and therefore can induce an antigen-specific immune response in an individual.

In some embodiments, the 5′-cap region of the messenger RNAs of the present invention may be a 5′ capping terminal, and ribonucleic acid caps may be used in an in vitro transcription reaction to generate a 5′-cap structure in the 5′-cap region. The 5′-cap structure may be, for example but not limited to, standard capping, an anti-reverse cap analogue (ARCA) or the commercially available CleanCap. The presence of the 5′-cap region is important to provide the messenger RNAs of the present invention with resistance to the nucleases found in most eukaryotic cells.

In the natural environment, usually a repetitive sequence may be added to immature messenger RNAs in the modification step to show the characteristic structure of mature messenger RNAs, such as the poly-polyadenosine tail region (3′-poly-A tail) terminal region. The poly-A tail region is usually an extension consisting of adenine nucleotides added to the 3′-terminus of the transcribed messenger RNAs. A poly-A tail typically contains about 10 to 300 adenine nucleotides, or may typically contain up to about 400 adenine nucleotides. In some embodiments, the length of the poly-A tail is a necessary element relative to the desired stability of the individual messenger RNAs. The presence of a poly-A tail region protects mature messenger RNAs from the degradation by exonuclease. In other embodiments, the 3′-poly-A region of the messenger RNAs of the present invention may be, for example, the sequence of SEQ ID NO: 9 or SEQ ID NO: 16, but the present invention is not limited thereto.

In some embodiments, the messenger RNAs of the present invention may include one or more un-translated regions. Untranslated regions help stabilize messenger RNAs. For example, it has been found that naturally-occurring eukaryotic messenger RNA molecules may include other stabilizing regions in addition to structural features such as a 5′-end cap structure or a 3′-poly-A tail region, for example but not limited to, one or more segments of untranslated regions (UTRs). One or more segments of untranslated regions may include a 5′-untranslated region (5′UTR) adjacent to the 5′ end cap region, and/or a 3′-untranslated region (3′UTR) adjacent to the 3′-poly-A tail region. Both the 5′-untranslated region and the 3′-untranslated region are generally necessary elements relative to the desired stability of the individual messenger RNAs.

In some embodiments, the 5′-untranslated region of the messenger RNAs of the present invention may be, for example, the sequences of SEQ ID NO: 3, SEQ ID NO: 5, SEQ ID NO: 13, or SEQ ID NO: 16, but the present invention is not limited thereto. In other embodiments, the 3′-untranslated region of the messenger RNAs of the present invention may be, for example, the sequences of SEQ ID NO: 8 or SEQ ID NO: 18, but the present invention is not limited thereto.

One or more polynucleotides of the present invention may be encapsulated or packed in a pharmaceutically acceptable vector. In some embodiments, the pharmaceutically acceptable vector may also be referred to as a delivery vehicle, for example, may be a kind of nanoparticles. In some embodiments, the pharmaceutically acceptable vector may include the group consisting of lipid nanoparticles and polymeric nanoparticles. Lipid nanoparticles may include biodegradable lipid, ionizable lipid, cation lipid, helper lipid, and cholesterol, but the present invention is not limited thereto. The polymer nanoparticles may include biodegradable cation polymers, poly(β-aminoester) and poly(α-aminoester), but the present invention is not limited thereto. In some embodiments, the messenger RNA vaccines of the present invention may be delivered by using smaller lipid nanoparticles. The lipid nanoparticles may have a diameter of not greater than 150 nanometers, but the present invention is not limited thereto.

In some embodiments, the messenger RNAs of the present invention may include one or more coding regions. A coding region may include a gene of interest, and a ligand sequence encoding a CD40 ligand. Optionally, a coding region may further include a functional sequence encoding a viral replicase. This functional sequence encoding a viral replicase facilitates the self-amplification of a small amount of a messenger RNA vaccine of the present invention into a large amount of a messenger RNA vaccine.

In one embodiment of the invention, the gene of interest may include a gene encoding a virus protein. In some embodiments, the gene of interest in the present invention may include, for example, the sequences of SEQ ID NO: 6 or SEQ ID NO: 15, but the present invention is not limited thereto. The virus may be SARS-related coronavirus, but the present invention is not limited thereto. The SARS-related coronaviruses may be, for example but not limited to, SARS-CoV and SARS-CoV-2. For example, the gene of interest may include at least one of the 51 subunit and the S2 subunit of the SARS-CoV-2 spike protein, but the present invention is not limited thereto.

A SARS-related coronavirus is a human-infecting coronavirus of the Coronaviridae family. For example, SARS-CoV-2 can invade a human body through the human upper respiratory tract to achieve the infection by targeting the angiotensin-converting enzyme 2 expressed on the surface of various cells as a receptor. This virus particle contains a variety of major proteins, and among which the S1 protein of the receptor-binding domain of the spike protein can bind to the angiotensin-converting enzyme 2 before it invades human cells.

The spike protein of SARS-CoV-2 can include two subunits, such as the S1 protein and the S2 protein. For example, the two subunits can include specific sequences of the domains of the spike protein, the receptor-binding domain (RBD) of the spike protein or the receptor-binding motif (RBM) of its spike protein. The spike protein of this new coronavirus can include an amino acid sequence of 1-1213 or the corresponding ribonucleic acid sequence, and the sequences of these spike proteins or receptor-binding domains are known.

Known S1 proteins can include the amino acid sequence of 1-674. In one embodiment of the present invention, the gene of interest may include a sequence of interest encoding a virus spike protein or a receptor-binding domain, for example, may include the sequence of interest encoding a spike protein of a severe acute respiratory syndrome-related coronavirus or its receptor-binding domain, but the present invention is not limited thereto. For the ribonucleic acid sequence of the spike protein of SARS-CoV-2 or for the ribonucleic acid sequence of the receptor-binding domain of its spike protein, please refer to the published sources https://www.uniprot.org/uniprot/P0DTC2 (UniProtKB—P0DTC2).

In addition to the wild type of SARS-CoV-2, there are currently a variety of known mutants. A virus mutant may also be referred to as a variant or a variant strain. A variant strain may, for example, refer to a mutation resulting from one or more base changes, such as deletions, duplications or insertions of one or more bases of the virus. A variation in the nucleic acid sequence of a virus may lead to a change in viral antigens, also known as an antigenic drift. Antigenic drift may allow the mutated virus to escape the host's immune response, thereby affecting the efficacy of vaccines. This phenomenon of escaping the host's immune response is called immune escape. For example, certain mutations in the spike protein of SARS-CoV-2 may result in higher affinity to cellular receptors. The wild type may be, for example, strain Wuhan-Hu-1, and mutant strains for example but not limited to, variant strain B.1.1.7, variant strain B.1.1.28, or variant strain B.1.351.

A CD40 ligand (CD40L) is a surface protein to be expressed on T cells, such as on the surface of activated T cells. A CD40 ligand can bind to CD40 molecules on antigen presenting cells (APC) and cause B cell activation. The interaction of a CD40 ligand with a CD40 molecule can enhance cellular immune responses and humoral immune responses, thereby promoting the production of antibodies. In other words, the presence of a CD40 ligand is beneficial for the enhancement of the induction of an antigen-specific immune response in an individual.

In one embodiment of the present invention, the coding region of the messenger RNAs of the present invention may include a sequence encoding a CD40 ligand, for example but not limited to, the sequences of SEQ ID NO:7 or SEQ ID NO:17. Where the coding region includes a sequence encoding a CD40 ligand, the messenger RNAs of the present invention can advantageously express the antigenic protein of a virus and strengthen the induction of an antigen-specific immune response in an individual. That is, in the presence of a sequence encoding a CD40 ligand, it is advantageous for the messenger RNAs of the present invention to produce in an individual a sufficient amount of specific antibodies with respect to an antigen-specific immune response, for example but not limited to, one or more immunoglobulins of the IgG class. For the sequences encoding the CD40 ligand, please refer to the published source https://www.uniprot.org/uniprot/P29965 (UniProtKB—P29965).

In one embodiment of the present invention, the coding region of the messenger RNAs of the present invention may further include a functional sequence encoding a viral replicase. For example, alphaviruses in the Togaviridae family have a sequence of a viral replicase which is a nonstructural protein. This viral replicase can mediate the replication of RNAs, so it can advantageously amplify the genome of the target sequence to a large amount, and accordingly amplify an originally small amount of the target sequence, so as to express a large amount of the foreign protein encoded by the target sequence. In the absence of sequences encoding structural proteins, individual assemble of infectious virus particles is impossible even with sequences encoding non-structural proteins, which means the impossibility of re-infection of cells. By taking advantage of this feature, the alphavirus genome with defected sequence may be used as a carrier for a large amount of exogenous protein expression. Common alpha viruses include Venezuelan equine encephalitis virus, Semliki Forest virus and Chikungunya virus, but the present invention is not limited thereto. For the RNA sequences of viral replicase encoding non-structural protein, please refer to SEQ ID NO: 4, SEQ ID NO: 5, SEQ ID NO: 14 and SEQ ID NO: 16.

Generally speaking, the non-structural regions in the genome of alphaviruses can encode four non-structural protein, nsP1, nsP2, nsP3 and nsP4, and can produce corresponding functions including RNA replicase. For example, non-structural regions of the genome of alphaviruses may correspond to non-structural protein RNA sequences, and such non-structural protein RNA sequences may be located between one or more non-translated regions of the messenger RNAs of the present invention, such as between the 5′ untranslated region and the 3′ untranslated region. When the non-structural protein RNA sequence is used as a functional sequence, a coding region containing a gene of interest may be arranged between the non-structural protein RNA sequence and the 3′ untranslated region to facilitate the insertion of exogenous RNA sequences and facilitate the promotion of expression of foreign genes.

In some embodiments of the present invention, the sequences of the non-structural protein and/or the sequences of the gene of interest may be optionally inserted into the sequence of the 5′-untranslated region to obtain a result of an insertional combination. For example, SEQ ID NO: 5 demonstrates a result of inserting the sequence of the non-structural protein into the sequence of the 5′-untranslated region, or SEQ ID NO: 16 demonstrates a result of respectively inserting the sequence of the non-structural protein and the sequence of the gene of interest, one after the other, into the same 5′-untranslated region, but the present invention is not limited thereto.

Since the viral replicase can mediate the replication of RNAs, and can amplify the genome of the target sequence in a large amount, in one embodiment of the present invention, the coding region in the polynucleotide of the present invention may also include a functional sequence, for example, include a self-amplifying functional sequence, but the present invention is not limited thereto. The functional sequence including the viral replicase may be equipped with the function of self-amplifying target RNA replication, so the messenger RNA vaccines of the present invention may be optimized to become a self-amplifying messenger RNA vaccine (SAM).

SEQ ID NO: 4 or SEQ ID NO: 14 respectively exemplifies a self-amplifying functional sequence suitable for use in the messenger RNA vaccines of the present invention. In some embodiments of the present invention, the self-amplifying functional sequence of the present invention, alone or together with the gene of interest, may be optionally inserted into the sequence of the 5′-untranslated region, to obtain a result of an insertional combination. SEQ ID NO: 5 exemplifies a result of an insertional combination in which the self-amplifying functional sequence suitable for the present invention is optionally inserted into the sequence of the 5′-untranslated region individually, but the present invention is not limited thereto. Alternatively, SEQ ID NO: 16 exemplifies a result of an insertional combination in which the self-amplifying functional sequence suitable for the present invention, together with the gene of interest, is optionally one after the other inserted into the sequence of the 5′-untranslated region, but the present invention is not limited thereto.

Several experimental examples are provided below to demonstrate the advantages of the present invention, but they are not intended to limit the scope of the present invention. Those of ordinary skill in the art to which the present invention pertains may make various changes and modifications without departing from the gist and scope of the present invention, which are still considered to be within the scope of the present invention.

The following provides the processes and results of the antibody kinetic assays according to the messenger RNA vaccines of the present invention.

### Experiment 1

RBD SAM LNP and S1+S2 SAM LNP Induce High Titer Antibody Response Against SARS-CoV-2 Protein

Materials:

### Messenger RNA Vaccine

1. As shown in SEQ ID NO: 11, self-amplifying messenger RNA sequences of lipid nanoparticles of the receptor-binding domain of severe acute respiratory syndrome coronavirus 2, with pig codon optimization, with the gene of interest to be the receptor-binding domain and without CD40 ligand;
2. As shown in SEQ ID NO: 12, self-amplifying messenger RNA sequences of lipid nanoparticles of the receptor-binding domain of severe acute respiratory syndrome coronavirus 2, with pig codon optimization, with the gene of interest to be the receptor-binding domain and with CD40 ligand;
3. As shown in SEQ ID NO: 1, self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and without CD40 ligand;
4. As shown in SEQ ID NO: 2, self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and with CD40 ligand;
5. As shown in SEQ ID NO: 10, self-amplifying messenger RNA sequences of lipid nanoparticles of the spike protein S1+S2 of severe acute respiratory syndrome coronavirus 2, with the gene of interest to be the S1+S2 of the spike protein and alphavirus;
6. The immunopurified polyclonal antibody (pAb) of SARS-nCoV receptor-binding domain protein with Fc tag as a control group.

Raw Materials:

1. RNase-free 10× phosphate buffered saline
2. RNase-free water

Equipment:

1. Insulin needles

### 2. Micropipette P100

### 3. Micropipette P20

Animals:

BALB/c mice (M02)

Experiment Procedures:

Taken out of the refrigerator, the self-amplifying messenger RNA vaccine was thawed on ice for 10 minutes. Later, 11 μg of the self-amplifying messenger RNA vaccine was taken out with the volume 11 μL, 8.8 μL of RNase-free 10× phosphate buffered saline and 68.5 mL of RNase-free water were added to, and they were mixed slowly and uniformly with a micropipette to have a total volume of 88 μL (the total volume remains unchanged, and the administration of different dosages are done by adjusting the total volume of the messenger RNA vaccines and RNase-free water). Mice were grabbed for intramuscular injection by the hind thighs on both sides with each injection volume of 40 μL. After the injection, the vital signs and activity of the mice were continuously observed. Doses were administered on the 0th day and the 28^{th} day respectively. The sera of multiple strains of mice were collected on the 35^{th} day for the evaluation of the antibody by enzyme-linked immunosorbent assay (ELISA).

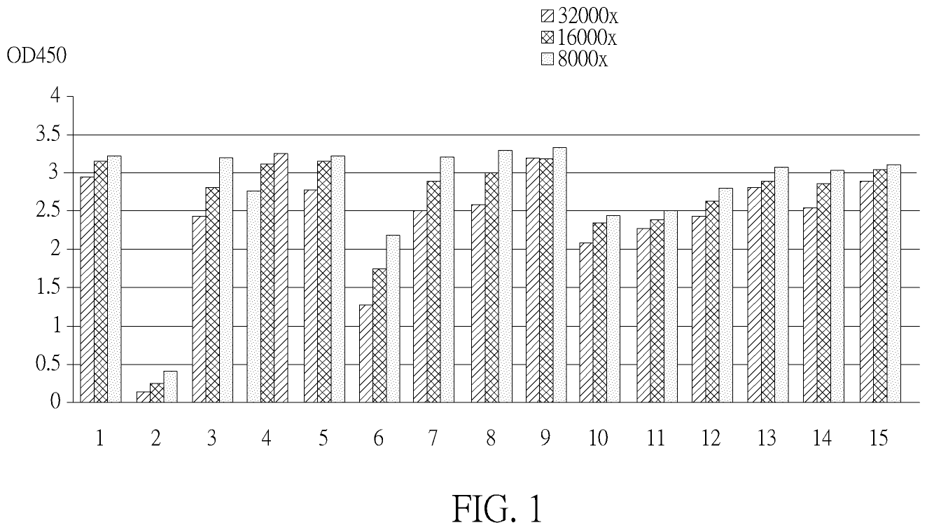

Please refer to FIG. 1. FIG. 1 shows the analysis data of the absorbance at 450 nm using the self-amplifying messenger RNA vaccine of the present invention by enzyme-linked immunosorbent assay. The X-axis represents the experimental and control groups of various combinations of polynucleotides, and the Y-axis OD450 represents the absorbance at 450 nm.

The combinations of polynucleotides in FIG. 1 are respectively: 1. the immunopurified polyclonal antibody with Fc-tagged with respect to SARS-nCoV receptor-binding domain protein as a control group; 2. a result of a dosage of 0.1 μg, with pig codon optimization, with the gene of interest to be the receptor-binding domain, without CD40 ligand; 3. a result of a dosage of 1 μg, with pig codon optimization, with the gene of interest to be the receptor-binding domain, without CD40 ligand; 4. a result of a dosage of 5 μg, with pig codon optimization, with the gene of interest to be the receptor-binding domain, without CD40 ligand; 5. a result of a dosage of 20 μg, with pig codon optimization, with the gene of interest to be the receptor-binding domain, without CD40 ligand; 6. a result of a dosage of 0.1 μg, with pig codon optimization, with the gene of interest to be the receptor-binding domain, with CD40 ligand; 7. a result of a dosage of 1 μg, with pig codon optimization, with the gene of interest to be the receptor-binding domain, with CD40 ligand; 8. a result of a dosage of 5 μg, with pig codon optimization, with the gene of interest to be the receptor-binding domain, with CD40 ligand; 9. a result of a dosage of 20 μg, with pig codon optimization, with the gene of interest to be the receptor-binding domain, with CD40 ligand; 10. a result of a dosage of 5 μg, with human codon optimization, with anti-kanamycin, with the gene of interest to be the receptor-binding domain, without CD40 ligand; 11. a result of a dosage of 20 μg, with human codon optimization, with anti-kanamycin, with the gene of interest to be the receptor-binding domain, without CD40 ligand; 12. a result of a dosage of 5 μg, with human codon optimization, with anti-kanamycin, with the gene of interest to be the receptor-binding domain, with CD40 ligand; 13. a result of a dosage of 20 μg, with human codon optimization, with anti-kanamycin, with the gene of interest to be the receptor-binding domain, with CD40 ligand; 14. a result of a dosage of 5 μg, with the gene of interest to be alphavirus and S1+S2 of the spike protein; 15. a result of a dosage of 20 μg, with the gene of interest to be alphavirus and S1+S2 of the spike protein. 32000×, 16000×, and 8000× represent the dilution ratios, respectively.

The experimental results show that except 1, other groups of 3-15 exhibit sufficient OD450 absorbance, that is, they can produce antibodies of high-efficiency. In particular, 2 compared with 6, 6 of the same low dose can still induce higher titers of antigen-specific immune responses at high dilution ratios. That is to say, the CD40 ligand introduced in the present invention is beneficial to induce the antigen-specific immune response, thereby increasing the titers of the produced antibody.

### Experiment 2

Comparison of Post-Immunization Mouse Sera Antibody Titers

Materials:

1. Mouse sera obtained from experiment 1 after administration:
(1) 5 μg or 20 μg mouse serum—self-amplifying messenger RNA sequences of lipid nanoparticles of the receptor-binding domain of severe acute respiratory syndrome coronavirus 2, with pig codon optimization, with the gene of interest to be the receptor-binding domain and without CD40 ligand;
(2) 5 μg or 20 μg mouse serum—self-amplifying messenger RNA sequences of lipid nanoparticles of the receptor-binding domain of severe acute respiratory syndrome coronavirus 2, with pig codon optimization, with the gene of interest to be the receptor-binding domain and with CD40 ligand;
(3) 5 μg or 20 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and without CD40 ligand;
(4) 5 μg or 20 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and with CD40 ligand;
(5) 5 μg or 20 μg mouse serum—self-amplifying messenger RNA sequences of lipid nanoparticles of the S1+S2 of the spike protein of severe acute respiratory syndrome coronavirus 2, with the gene of interest to be the alphavirus and S1+S2 of the spike protein;
2. Pseudovirus: SARS-CoV-2 pseudovirus expressing firefly luciferase;
3. Cell line: HEK-293T cells expressing human ACE2, abbreviated as 293T-ACE2 cells, from ABNOVA;
4. Luminescent reagent.

Experimental Procedures:

The same as described in experiment 1.

Sera antibody titers are compared as follows:

10 μL of SARS-CoV-2 pseudovirus was mixed with a mouse serum (4-fold serial dilution), and the pseudovirus and serum mixture was kept at room temperature for 30 minutes. After the pseudovirus and serum mixture was added to 293T-ACE2 cells (1×10⁵ cells/24-well plate) and placed in a 5% carbon dioxide 37° C. incubator for 48 hours, firefly luminase activity was measure. The 24-well plate was taken out from the incubator, 150 μL of a passive lysis buffer was added to each well and kept at room temperature for 20 minutes. 10 μL of a lysed sample was taken out with a microdispenser and added to an optiplate 96-well plate. After 50 μL of a firefly luciferase buffer (containing firefly luciferase 1:50) was added, a luminometer was used to detect the luminescence signals.

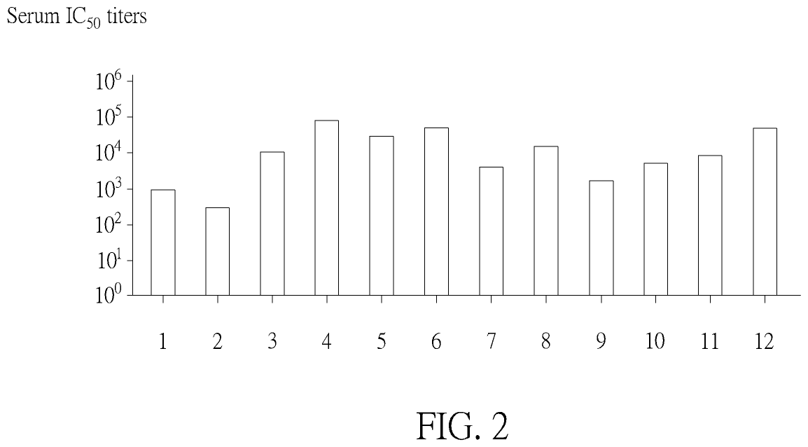

Please refer to FIG. 2. FIG. 2 shows comparison data of the serum IC₅₀ titers between conventional messenger RNA vaccines and the messenger RNA vaccines of the present invention. The X axis represents the experimental group and the control group of various messenger RNA vaccines, and the Y axis represents the serum IC₅₀ titers.

The messenger RNA vaccines in FIG. 2 are respectively: 1-2 are commercially available messenger RNA vaccines as the control groups. 3-12 are the messenger RNA vaccines of the present invention as the experimental groups. 1. is the data of 1 μg of Moderna mRNA-1273 vaccine, obtained from SARS-CoV-2 mRNA Vaccine Development Enabled by Prototype Pathogen Preparedness, 2020 Jun. 11. 2. is the data of 5 μg of BioNTech/Pfizer's BNT162b2 vaccine, obtained from A prefusion SARS-CoV-2 Spike RNA Vaccine is highly immunogenic and prevents lung infection in non-human primates, 2020 Sep. 8. 3. the data of a dosage of 5 μg, with pig codon optimization, with the gene of interest to be the receptor-binding domain, without CD40 ligand; 4. the data of a dosage of 20 μg, with pig codon optimization, with the gene of interest to be the receptor-binding domain, without CD40 ligand; 5. the data of a dosage of 5 μg, with pig codon optimization, with the gene of interest to be the receptor-binding domain, with CD40 ligand; 6. the data of a dosage of 20 μg, with pig codon optimization, with the gene of interest to be the receptor-binding domain, with CD40 ligand; 7. the data of a dosage of 5 μg, with the gene of interest to be alphavirus and S1+S2 of the spike protein; 8. the data of a dosage of 20 μg, with the gene of interest to be alphavirus and S1+S2 of the spike protein; 9. the data of a dosage of 5 μg, with human codon optimization, with anti-kanamycin, with the gene of interest to be the receptor-binding domain, without CD40 ligand; 10. the data of a dosage of 20 μg, with human codon optimization, with anti-kanamycin, with the gene of interest to be the receptor-binding domain, without CD40 ligand; 11. the data of a dosage of 5 μg, with human codon optimization, with anti-kanamycin, with the gene of interest to be the receptor-binding domain, with CD40 ligand; 12. the data of a dosage of 20 μg, with human codon optimization, with anti-kanamycin, with the gene of interest to be the receptor-binding domain, with CD40 ligand.

The experimental results show significantly higher titers of the messenger RNA vaccines of the present invention in 3-12 experimental groups compared with the conventional messenger RNA vaccines as the 1-2 control groups. In addition, the antibody titers of the experimental groups of the 3-12 vaccines of the present invention are generally higher than 1000. In particular, at the same dosage of 5 μg, the antibody titers of the vaccines in the experimental groups of the present invention are generally higher than those of the conventional BNT162b2 vaccine. Therefore, compared with the conventional messenger RNA vaccine, the inventive step of the messenger RNA vaccines of the present invention with significantly higher antibody titers is confirmed.

### Experiment 3

Correlation Between SARS-CoV-2 IgG and SARS-CoV-2 Pseudovirus Neutralization

Materials:

Pseudoviruses of SARS-CoV-2, B.1.1.7 and B.1.351 variant pseudoviruses, messenger RNA vaccines of various combinations of the present invention

1. Mouse sera obtained from experiment 1 administration:
(1) 5 μg mouse serum—self-amplifying messenger RNA sequences of lipid nanoparticles of the receptor-binding domain of severe acute respiratory syndrome coronavirus 2, with pig codon optimization, with the gene of interest to be the receptor-binding domain and without CD40 ligand;
(2) 20 μg mouse serum—self-amplifying messenger RNA sequences of lipid nanoparticles of the receptor-binding domain of severe acute respiratory syndrome coronavirus 2, with pig codon optimization, with the gene of interest to be the receptor-binding domain and without CD40 ligand;
(3) 5 μg mouse serum—self-amplifying messenger RNA sequences of lipid nanoparticles of the receptor-binding domain of severe acute respiratory syndrome coronavirus 2, with pig codon optimization, with the gene of interest to be the receptor-binding domain and with CD40 ligand;
(4) 20 μg mouse serum—self-amplifying messenger RNA sequences of lipid nanoparticles of the receptor-binding domain of severe acute respiratory syndrome coronavirus 2, with pig codon optimization, with the gene of interest to be the receptor-binding domain and with CD40 ligand;
2. Pseudovirus: pseudovirus expressing firefly luciferase
(1) SARS-CoV-2 pseudovirus;
(2) B.1.1.7 variant strain pseudovirus;
(3) B.1.351 variant strain pseudovirus;
3. Cell line: HEK-293T cells expressing human ACE2, abbreviated as 293T-ACE2 cells, from ABNOVA;
4. luciferase

Experimental Procedures:

Sera were drawn on the 35^{th}, 42^{nd}, 49^{th}, and 182^{nd} day after the mixed administration of pseudovirus of SARS-CoV-2, B.1.1.7 and B.1.351 variant strain pseudoviruses (10 μl each) (50-fold dilution, 4-folds serial dilution). The obtained sera were incubated at room temperature for 30 minutes. A mixture of pseudovirus and serum was added to 293T-ACE2 cells (1×10⁵ cells/24-well plate). Luciferase activity was detected 48 hours after pseudovirus transducing.

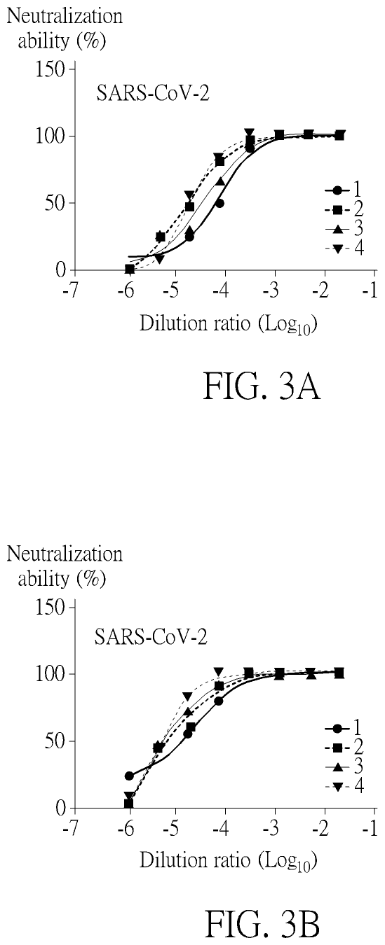

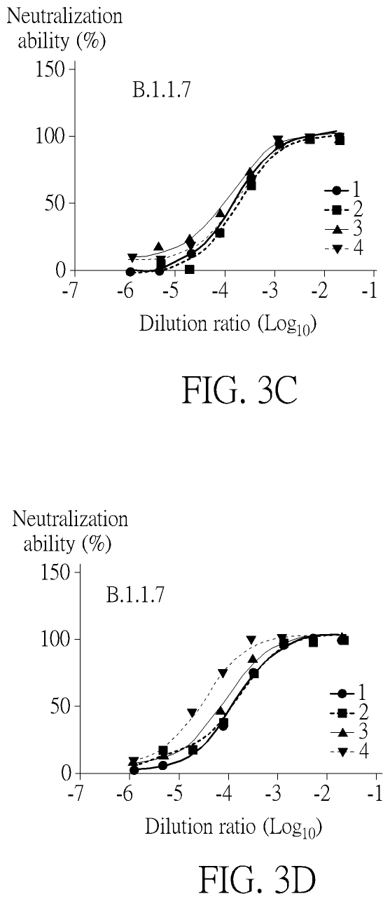

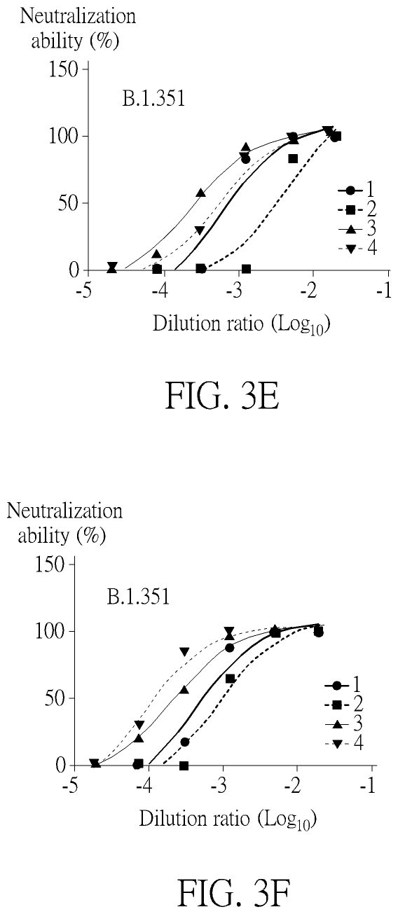

Please refer to FIG. 3A, FIG. 3B, FIG. 3C, FIG. 3D, FIG. 3E, FIG. 3F. FIG. 3A, FIG. 3B, FIG. 3C, FIG. 3D, FIG. 3E, FIG. 3F respectively show the neutralizing ability correlation data of the collected mouse sera for IgG immune responses to the receptor-binding domain 35 days and 42 days after the administration of the messenger RNA vaccines of the present invention. The X-axis represents the dilution ratio (log₁₀) of various sera, and the Y-axis represents the neutralization ability to the pseudovirus of SARS-CoV-2. FIG. 3A, FIG. 3C and FIG. 3E respectively show the data of the mouse sera collected 35 days after the administration. FIG. 3B, FIG. 3D and FIG. 3F respectively show the data of the mouse sera collected 42 days after the administration. FIG. 3A and FIG. 3B respectively show the data against the wild strain. FIG. 3C and FIG. 3D respectively show the data against the variant strain B.1.1.7. FIG. 3E and FIG. 3F respectively show the data against the variant strain B.1.351.

The messenger RNA vaccines in each figure are respectively:

1. the data of 5 μg with pig codon optimization, with the gene of interest to be the receptor-binding domain and without CD40 ligand;
2. the data of 20 μg with pig codon optimization, with the gene of interest to be the receptor-binding domain and without CD40 ligand;
3. the data of 5 μg with pig codon optimization, with the gene of interest to be the receptor-binding domain and with CD40 ligand; 4. the data of 20 μg with pig codon optimization, with the gene of interest to be the receptor-binding domain and with CD40 ligand.

The experimental results show that the messenger RNA vaccines of the present invention respectively exhibit excellent pseudovirus neutralization ability both at high (20 μg) or at low (5 μg) administered dosage. In addition, the messenger RNA vaccines of the present invention exhibit excellent pseudovirus neutralization ability after 35 days of administration, for example, it exhibits an excellent higher than 50% pseudovirus neutralization ability at a lower than 10⁻⁴ dilution ratio (FIG. 3A). Further, the messenger RNA vaccines of the present invention exhibit stronger pseudovirus neutralization ability 42 days after the administration, for example, up to an excellent higher than 50% pseudovirus neutralization ability at an even lower than about 10⁻⁵ dilution ratio (FIG. 3B). In particular, the messenger RNA vaccines of the present invention still exhibit excellent neutralizing ability even against pseudoviruses of multiple spike protein variant strains of SARS-CoV-2 (FIG. 3D). Therefore, the inventive step of the messenger RNA vaccines of the present invention with excellent pseudovirus neutralization ability against not only SARS-CoV-2 pseudoviruses but also against B.1.1.7 and B.1.351 variant strain pseudoviruses is confirmed.

### Experiment 4

Comparison of RBD Vs S1+S2 SAM Between SARS-CoV-2 and SARS-CoV-2 Variant Pseudovirus Neutralization

Materials:

1. Mouse sera obtained from experiment 1 administration:
(1) 5 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and without CD40 ligand;
(2) 20 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and without CD40 ligand;
(3) 5 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and with CD40 ligand;
(4) 20 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and with CD40 ligand;
(5) 5 μg mouse serum—self-amplifying messenger RNA sequences of lipid nanoparticles of S1+S2 of the spike protein of severe acute respiratory syndrome coronavirus 2, with the gene of interest to be the alphavirus and S1+S2 of the spike protein;
(6) 20 μg mouse serum—self-amplifying messenger RNA sequences of lipid nanoparticles of S1+S2 of the spike protein of severe acute respiratory syndrome coronavirus 2, with the gene of interest to be the alphavirus and S1+S2 of the spike protein;
2. Pseudovirus: pseudovirus expressing firefly luciferase
(1) SARS-CoV-2 pseudovirus;
(2) B.1.1.7 variant strain pseudovirus;
(3) B.1.351 variant strain pseudovirus;
3. Cell line: HEK-293T cells expressing human ACE2, abbreviated as 293T-ACE2 cells, from ABNOVA;
4. Luminescent reagent: Luciferase Assay System 10-Pack, E1501 Promega Luciferase Cell Culture Lysis 5× Reagent E1531 Promega

Experimental Procedures:

Sera were drawn on the 35^{th}, 42^{nd} day after the mixed administration of SARS-CoV-2 pseudovirus, B.1.1.7 and B.1.351 variant strain pseudoviruses (10 μl each) (50-fold dilution, 4-folds serial dilution). The obtained sera were incubated at room temperature for 30 minutes. A mixture of pseudovirus and serum was added to 293T-ACE2 cells (1×10⁵ cells/24-well plate). Luciferase activity was detected 48 hours after pseudovirus transducing.

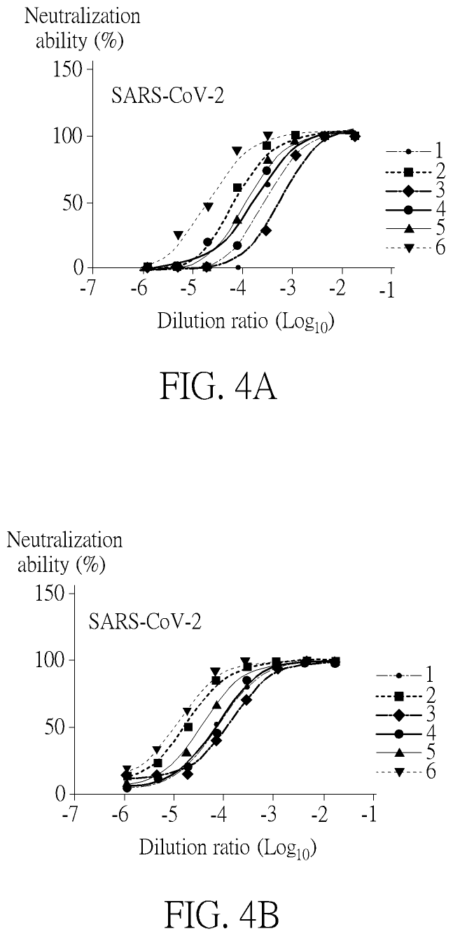

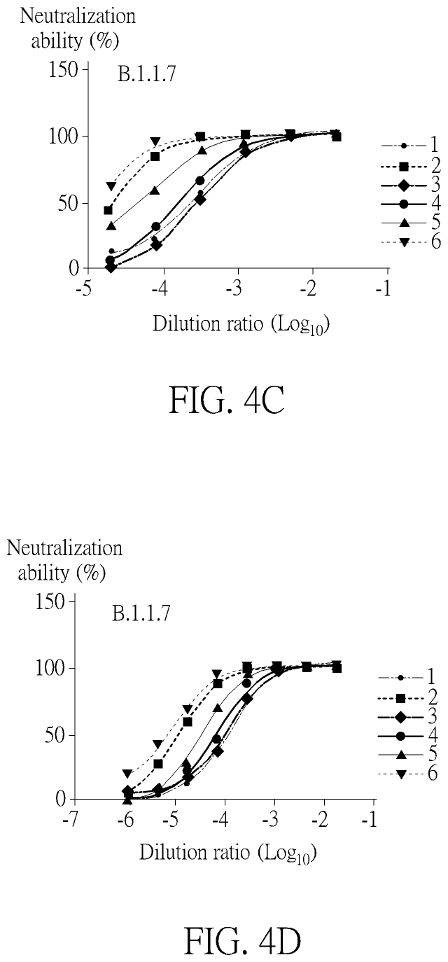

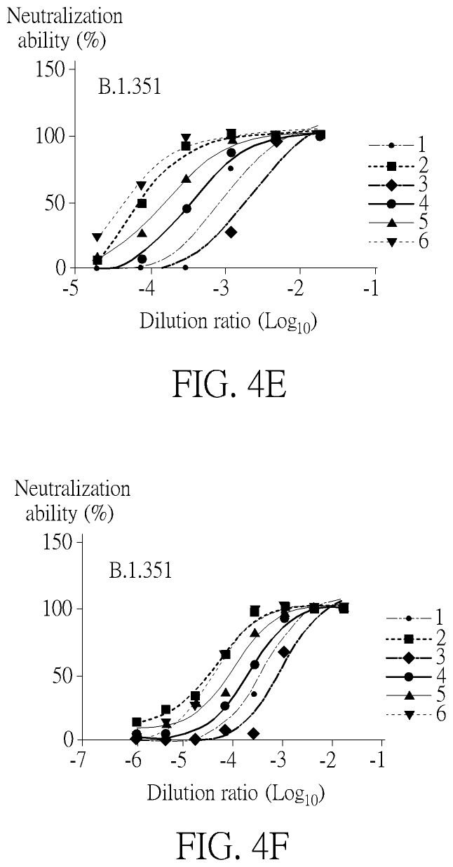

Please refer to FIG. 4A, FIG. 4B, FIG. 4C, FIG. 4D, FIG. 4E, and FIG. 4F. FIG. 4A, FIG. 4B, FIG. 4C, FIG. 4D, FIG. 4E, and FIG. 4F respectively show the neutralizing ability data of the collected mouse sera for IgG immune responses to the receptor-binding domain 35 days and 42 days after the administration of the messenger RNA vaccines of the present invention. The X-axis represents the dilution ratio (log₁₀) of various sera, and the Y-axis represents the neutralization ability to various pseudoviruses of SARS-CoV-2. FIG. 4A, FIG. 4C and FIG. 4E respectively show the data of the mouse sera collected 35 days after the administration. FIG. 4B, FIG. 4D and FIG. 4F respectively show the data of the mouse sera collected 42 days after the administration. FIG. 4A and FIG. 4B respectively show the data against the wild strain. FIG. 4C and FIG. 4D respectively show the data against the variant strain B.1.1.7. FIG. 4E and FIG. 4F respectively show the data against variant strain B.1.351.

The messenger RNA vaccines in each figure are respectively: 1. the data of a dosage of 5 μg, with the gene of interest to be S1+S2 of the spike protein and alphavirus; 2. the data of a dosage of 20 μg, with the gene of interest to be S1+S2 of the spike protein and alphavirus; 3. the data of a dosage of 5 μg, with human codon optimization, with anti-kanamycin, with the gene of interest to be the receptor-binding domain, without CD40 ligand; 4. the data of a dosage of 20 μg, with human codon optimization, with anti-kanamycin, with the gene of interest to be the receptor-binding domain, without CD40 ligand; 5. the data of a dosage of 5 μg, with human codon optimization, with anti-kanamycin, with the gene of interest to be the receptor-binding domain, with CD40 ligand; 6. the data of a dosage of 20 μg, with human codon optimization, with anti-kanamycin, with the gene of interest to be the receptor-binding domain, with CD40 ligand.

The experimental results show that the messenger RNA vaccines of the present invention respectively exhibit excellent pseudovirus neutralization ability both at high (20 μg) or at low (5 μg) administered dosage. In addition, the messenger RNA vaccines of the present invention exhibit an excellent higher than 50% pseudovirus neutralization ability at a lower than 10⁻⁴ dilution ratio (FIG. 4C) after 35 days of administration. Further, the messenger RNA vaccines of the present invention exhibit a higher than 50% pseudovirus neutralization ability at an even lower than about 10⁻⁵ dilution ratio (FIG. 4D) after 42 days of administration. In particular, the messenger RNA vaccines of the present invention still exhibit remaining excellent about 50% pseudovirus neutralization ability at a 10⁻⁵ dilution ratio (FIG. 4C) against pseudoviruses of multiple spike protein variant strains of SARS-CoV-2. Therefore, the inventive step of the messenger RNA vaccines of the present invention with excellent pseudovirus neutralization ability against not only SARS-CoV-2 pseudoviruses but also against B.1.1.7 and B.1.351 variant strains pseudoviruses is confirmed.

Please see Table 1. Table 1 shows the IC₅₀ titers of the pseudoviruses corresponding to 35 days or 42 days after the administration of the messenger RNA vaccines of the present invention in experiment 4.

TABLE 1
IC₅₀ titers of the pseudoviruses corresponding
to 35 days or 42 days after the administration of the
vaccines in experiment 4

| | Wild | Wild | B.1.1.7 | B.1.1.7 | B.1.351 | B.1.351 |
|---|---|---|---|---|---|---|
| Typesof | 35 | 42 | 35 | 42 | 35 | 42 |
| pseudovirus | days | days | days | days | days | days |
| IC₅₀ titers | | | | | | |
| 1 | 3675 | 11357 | 3840 | 7740 | 1110 | 2235 |
| 2 | 15939 | 47778 | 50000 | 85034 | 23364 | 18699 |
| 3 | 1744 | 5963 | 3812 | 7220 | 412 | 885 |
| 4 | 5596 | 11677 | 6631 | 12577 | 3129 | 4024 |
| 5 | 8795 | 24331 | 20000 | 26991 | 6545 | 8264 |
| 6 | 55617 | 76336 | 51200 | 102733 | 33212 | 22676 |

### Experiment 5

Comparison of PNP Vs LNP Deliveries Between SARS-CoV-2 and SARS-CoV-2 Variant Pseudovirus Neutralization

Materials:

1. Mouse sera obtained from experiment 1 administration:
(1) 5 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and with CD40 ligand;
(2) 5 μg mouse serum—self-amplifying messenger RNA sequences of polymer nanoparticles of the receptor-binding domain of severe acute respiratory syndrome coronavirus 2, with human codon optimization, with anti-kanamycin, with the gene of interest to be the receptor-binding domain and with CD40 ligand;
(3) 5 μg mouse serum—self-amplifying messenger RNA sequences of lipid nanoparticles of S1+S2 of the spike protein of severe acute respiratory syndrome coronavirus 2, with the gene of interest to be the alphavirus and S1+S2 of the spike protein;
(4) 5 μg mouse serum—self-amplifying messenger RNA sequences of polymer nanoparticles of S1+S2 of the spike protein of severe acute respiratory syndrome coronavirus 2, with the gene of interest to be the alphavirus and S1+S2 of the spike protein;
2. Pseudovirus: pseudovirus expressing firefly luciferase
(1) SARS-CoV-2 pseudovirus;
(2) B.1.1.7 variant strain pseudovirus;
(3) B.1.351 variant strain pseudovirus;
3. Cell line: HEK-293T cells expressing human ACE2, abbreviated as 293T-ACE2 cells, from ABNOVA;
4. Luminescent reagent: Luciferase Assay System 10-Pack, E1501 Promega Luciferase Cell Culture Lysis 5× Reagent E1531 Promega

Experimental Animals:

BALB/c mice (M02)

Experimental Procedures:

Immunization methods of lipid nanoparticle vaccines are the same as described in experiment 1.

Immunization methods of polymer nanoparticles vaccines are as follows:

Taken out of the refrigerator, the self-amplifying messenger RNA vaccine was thawed on ice for 10 minutes. Later, 11 μg of the self-amplifying messenger RNA vaccine was taken out with the volume 11 μL, 91.5 μL of acid phosphate buffered saline and 7.5 mL of polymer nanoparticles were added to, and they were mixed slowly and uniformly with a micropipette to have a total volume of 110 μL (the total volume remains unchanged, and the administration of different dosages are done by adjusting the total volume of the messenger RNA vaccines, the acid phosphate buffered saline and the polymer nanoparticles). Mice were grabbed for intramuscular injection in the hind thighs on both sides with each injection volume of 50 μL. After the injection, the vital signs and activity of the mice were continuously observed. Doses were administered on the 0^{th} day and the 28^{th} day respectively.

Serum antibody titers were compared as follows:

Sera were drawn on the 35^{th}, 42^{nd} day after the mixed administration of SARS-CoV-2 pseudovirus, B.1.1.7 and B.1.351 variant strain pseudoviruses (10 μl each) (50-fold dilution, 4-folds serial dilution). The obtained sera were incubated at room temperature for 30 minutes. A mixture of pseudovirus and serum was added to 293T-ACE2 cells (1×10⁵ cells/24-well plate). Luciferase activity was detected 48 hours after pseudovirus transducing.

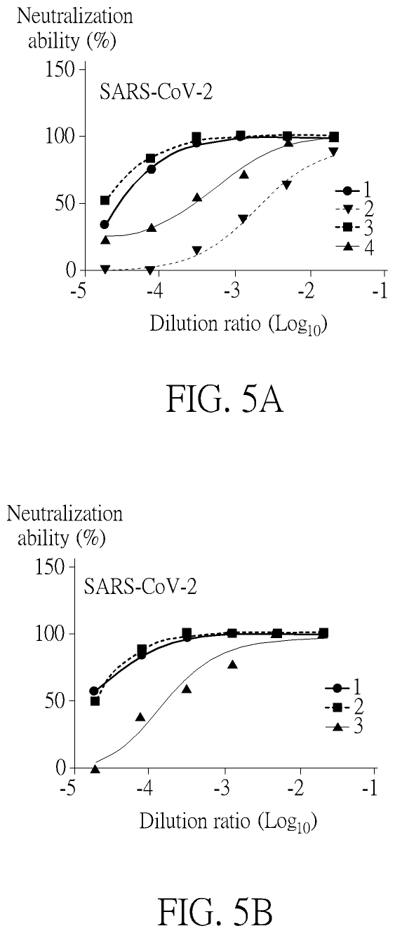

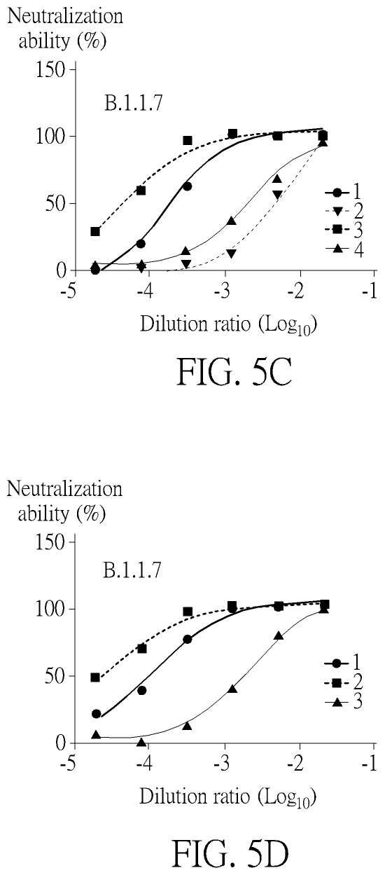

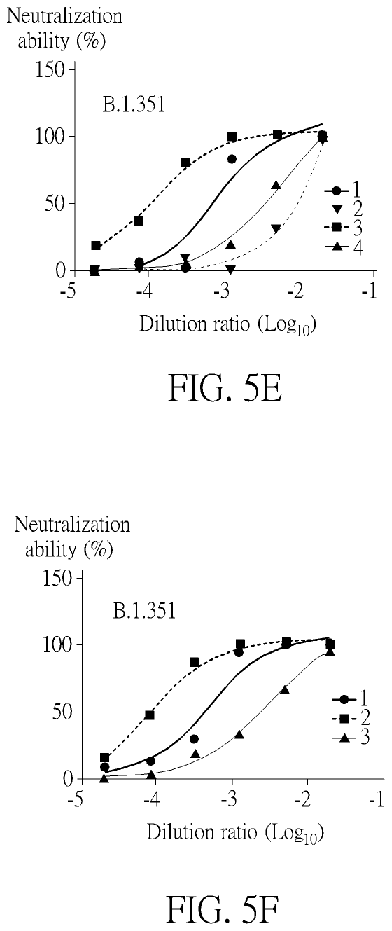

Please refer to FIG. 5A, FIG. 5B, FIG. 5C, FIG. 5D, FIG. 5E, and FIG. 5F. FIG. 5A, FIG. 5B, FIG. 5C, FIG. 5D, FIG. 5E, and FIG. 5F respectively show the neutralizing ability data of the collected mouse sera for IgG immune responses to the receptor-binding domain 35 days and 42 days after the administration of the messenger RNA vaccines of the present invention encapsulated in different vectors. The X-axis represents the dilution ratio (log₁₀) of various sera, and the Y-axis represents the neutralization ability to various pseudoviruses of SARS-CoV-2. FIG. 5A, FIG. 5C and FIG. 5E respectively show the data of the mouse sera collected 35 days after the administration. FIG. 5B, FIG. 5D and FIG. 5F respectively show the data of the mouse sera collected 42 days after the administration. FIG. 5A and FIG. 5B respectively show the data against the wild strain. FIG. 5C and FIG. 5D respectively show the data against the variant strain B.1.1.7. FIG. 5E and FIG. 5F respectively show the data against variant strain B.1.351.

The messenger RNA vaccines in FIG. 5A, in FIG. 5C and in FIG. 5E are respectively: 1. the data of a dosage of 5 μg, with the gene of interest to be S1+S2 of the spike protein and alphavirus, with LNP as the vector; 2. the data of a dosage of 5 μg, with the gene of interest to be S1+S2 of the spike protein and alphavirus, with PNP as the vector; 3. the data of a dosage of 5 μg, with human codon optimization, with anti-kanamycin, with the gene of interest to be the receptor-binding domain, with CD40 ligand, with LNP as the vector; 4. the data of a dosage of 5 μg, with human codon optimization, with anti-kanamycin, with the gene of interest to be the receptor-binding domain, with CD40 ligand, with PNP as the vector. The messenger RNA vaccines in FIG. 5B, in FIG. 5D and in FIG. 5F are respectively: 1. the data of a dosage of 5 μg, with the gene of interest to be S1+S2 of the spike protein and alphavirus, with LNP as the vector; 2. the data of a dosage of 5 μg, with human codon optimization, with anti-kanamycin, with the gene of interest to be the receptor-binding domain, with CD40 ligand, with LNP as the vector; 3. the data of a dosage of 5 μg, with human codon optimization, with anti-kanamycin, with the gene of interest to be the receptor-binding domain, with CD40 ligand, with PNP as the vector.

The experimental results show that the messenger RNA vaccines of the present invention respectively exhibit excellent pseudovirus neutralization ability both with PNP as the vector and with LNP as the vector. In addition, the messenger RNA vaccines of the present invention exhibit an excellent higher than 50% pseudovirus neutralization ability at a lower than 10⁻⁴ dilution ratio (FIG. 5A, FIG. 5C and FIG. 5E) after 35 days of administration with PNP as the vector. Further, the messenger RNA vaccines of the present invention maintain the pseudovirus neutralization ability after 42 days of administration. In particular, the messenger RNA vaccines of the present invention with LNP as the vector still exhibit excellent higher 50% pseudovirus neutralization ability at a lower 10⁻⁴ dilution ratio (FIG. 5A, FIG. 5B, FIG. 5C, FIG. 5D, FIG. 5E, and FIG. 5F) to pseudoviruses of multiple spike protein variant strains of SARS-CoV-2. Therefore, the inventive step of the messenger RNA vaccines of the present invention with LNP as the vector to exhibit excellent pseudovirus neutralization ability against not only SARS-CoV-2 pseudoviruses but also against B.1.1.7 and B.1.351 variant strains pseudoviruses is confirmed.

### Experiment 6

Comparison of Day 49 and Day 182 SARS-CoV-2 Pseudovirus Neutralization

Materials:

1. Mouse sera obtained from experiment 1 after administration:
(1) 5 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and without CD40 ligand;
(2) 20 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and without CD40 ligand;
(3) 5 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and with CD40 ligand;
(4) 20 μg mouse serum—self-amplifying messenger RNA sequences of lipid nanoparticles of the receptor-binding domain of severe acute respiratory syndrome coronavirus 2, with human codon optimization, with anti-kanamycin, with the gene of interest to be the receptor-binding domain and with CD40 ligand;
2. Pseudovirus: pseudovirus expressing firefly luciferase
(1) SARS-CoV-2 pseudovirus;
3. Cell line: HEK-293T cells expressing human ACE2, abbreviated as 293T-ACE2 cells, from ABNOVA;
4. Luminescent reagent: Luciferase Assay System 10-Pack, E1501 Promega Luciferase Cell Culture Lysis 5× Reagent E1531 Promega

Experimental Procedures:

Sera were drawn on the 49^{th}, 182^{nd} day after the mixed administration of pseudovirus of SARS-CoV-2 (10 μl each) (50-fold dilution, 4-folds serial dilution). The obtained sera were incubated at room temperature for 30 minutes. A mixture of pseudovirus and serum was added to 293T-ACE2 cells (1×10⁵ cells/24-well plate). Luciferase activity was detected 48 hours after pseudovirus transducing.

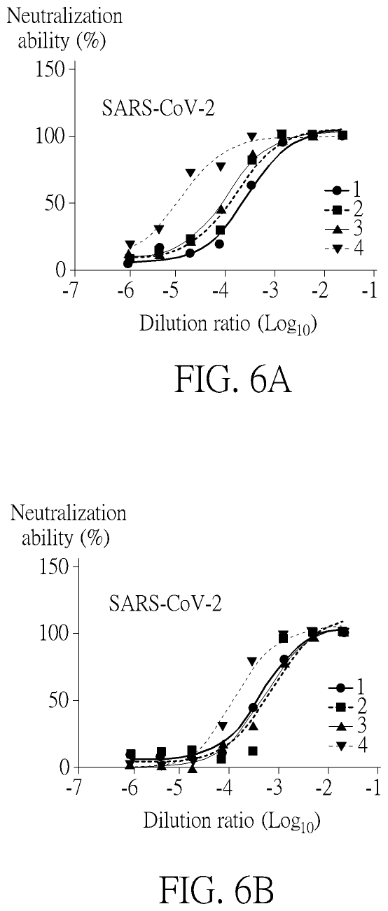

Please refer to FIG. 6A and FIG. 6B. FIG. 6A shows the neutralizing ability data of the collected mouse sera for IgG immune responses to the receptor-binding domain 49 days after the administration of the messenger RNA vaccines of the present invention. FIG. 6B shows the neutralizing ability data of the collected mouse sera for IgG immune responses to the receptor-binding domain 182 days after the administration of the messenger RNA vaccines of the present invention. The X-axis represents the dilution ratio (log₁₀) of the serum, and the Y-axis represents the neutralization ability to the pseudovirus of SARS-CoV-2.

The messenger RNA vaccines in FIG. 6A and in FIG. 6B are respectively: 1. the data of 5 μg with pig codon optimization, with the gene of interest to be the receptor-binding domain and without CD40 ligand; 2. the data of 20 μg with pig codon optimization, with the gene of interest to be the receptor-binding domain and without CD40 ligand; 3. the data of 5 μg with pig codon optimization, with the gene of interest to be the receptor-binding domain and with CD40 ligand; 4. the data of 20 μg with pig codon optimization, with the gene of interest to be the receptor-binding domain and with CD40 ligand.

The experimental results show that the messenger RNA vaccines of the present invention respectively exhibit excellent pseudovirus neutralization ability 49 days or 182 days after the administration. In addition, the messenger RNA vaccines with CD40 ligand of the present invention exhibit excellent pseudovirus neutralization ability 49 days after the administration, for example, they exhibit excellent up to 50% pseudovirus neutralization ability at a 10⁻⁵ dilution ratio (FIG. 6A). Further, the messenger RNA vaccines of the present invention may still maintain 50% pseudovirus neutralization ability at a 10⁻⁴ dilution ratio 182 days after the administration.

Please refer to Table 2. Table 2 shows the IC₅₀ titers of the pseudoviruses corresponding to 49 days or 182 days after the administration of the messenger RNA vaccines of the present invention in experiment 6.

TABLE 2
IC₅₀ titers of the pseudoviruses corresponding
to 49 days or 182 days after the administration
of the vaccines in experiment 6

| IC₅₀ titers of experimental groups | 49 days | 182 days |
|---|---|---|
| 1 | 3795 | 1999 |
| 2 | 6707 | 1304 |
| 3 | 9911 | 1729 |
| 4 | 81699 | 7003 |

### Experiment 7

Comparison of In Vivo Toxicity

Materials:

The messenger RNA vaccines used in this experiment are: a dosage of 5 μg, with the gene of interest to be S1+S2 of the spike protein and alphavirus, with PNP as the vector; a dosage of 20 μg, with the gene of interest to be S1+S2 of the spike protein and alphavirus, with PNP as the vector; a dosage of 5 μg, with human codon optimization, with anti-kanamycin, with the gene of interest to be the receptor-binding domain, with CD40 ligand, with polymer nanoparticles as the vector; a dosage of 20 μg, with human codon optimization, with anti-kanamycin, with the gene of interest to be the receptor-binding domain, with CD40 ligand, with polymer nanoparticles as the vector.

Experimental Animals:

BALB/c mice (M02)

Experimental Procedures:

Taken out of the refrigerator, the self-amplifying messenger RNA vaccine was thawed on ice for 10 minutes. Later, 11 μg of the self-amplifying messenger RNA vaccine was taken out with the volume 11 μL, 91.5 μL of acid phosphate buffered saline and 7.5 mL of polymer nanoparticles were added to, and they were mixed slowly and uniformly with a micropipette to have a total volume of 110 μL (the total volume remains unchanged, and the administration of different dosages are done by adjusting the total volume of the messenger RNA vaccines, the acid phosphate buffered saline and the polymer nanoparticles). Mice were grabbed for intramuscular injection by the hind thighs on both sides with each injection volume of 50 μL. After the injection, the vital signs and activity of the mice were continuously observed. Doses were administered on the 0^{th} day and the 28^{th} day respectively.

Please refer to Table 3. Table 3 lists the individual histopathologic findings of various tissues after the administration of various representative messenger RNA vaccines of the present invention. The severity grading scheme: 1=minimal (<10%), 2=mild (10-39%), 3=moderate (40-79%), 4=marked (80-100%). The symbol (-) indicates no abnormal findings.

TABLE 3
Individual histopathologic findings after the administration
of the messenger RNA vaccines of the present invention

| | | | | 20 μg, human |
|---|---|---|---|---|
| | | | 5 μg, | codon |
| | | | human codon | optimization, |
| Columns: | | | optimization, | anti-kanamycin, |
| types of | | | anti-kanamycin, | gene of |
| vaccines | | | gene of interest | interest |
| Rows: | | | receptor-binding | receptor-binding |
| histopatho- | | | domain, | domain, with |
| logically | 5 μg, | 20 μg, | with CD40 | CD40 ligand, |
| observed | S1 + S2, | S1 + S2, | ligand, polymer | polymer |
| tissues | PNP | PNP | nanoparticles | nanoparticles |
| Heart | — | — | — | 2 |
| Hemorrhage, | | | | |
| focal | | | | |
| Liver | — | — | 1 | — |
| Infiltration, | | | | |
| mononuclear | | | | |
| cell, focal | | | | |
| Spleen | — | — | — | — |
| Lung | — | 1 | — | — |
| Infiltration, | | | | |
| mononuclear | | | | |
| cell, focal | | | | |
| Kidneys | 1 | — | | — |
| Basophilia, | | | | |
| tubule, | | | | |
| cortex, focal | | | | |
| Dilation, | | 2 | | |
| tubule, | | | | |
| cortex, focal | | | | |
| Adrenal gland | 2 | 2 | 2 | 3 |
| Hyperplasia, | | | | |
| subcapsular, | | | | |
| cortex, focal | | | | |
| Brain | — | — | — | — |
| Thymus | | | | |
| Lymph nodes, | — | — | — | — |
| mandibular | | | | |
| Lymph nodes, | — | — | — | — |
| mesenteric | | | | |

According to the histopathology examinations, there were no observable toxic evidences of the tested self-amplifying messenger RNA vaccines of the present invention.

All data generated from the study would provide safety criteria information for human exposure.

The above histopathological examination results confirm that no evidence of toxicity can be found to the tested tissues after the administration of the representative self-amplifying messenger RNA vaccines of the present invention.

Those skilled in the art will readily observe that numerous modifications and alterations of the mRNA vaccine and method may be made while retaining the teachings of the invention. Accordingly, the above disclosure should be construed as limited only by the metes and bounds of the appended claims.

What is claimed is:

1. A messenger ribonucleic acid (mRNA) vaccine, comprising:
at least one ribonucleic acid (RNA) polynucleotide, each of the at least one polynucleotide comprising:
5′ end cap region;
an untranslated region (UTR);
a coding region comprising a gene of interest (GOI) and a ligand sequence encoding a CD40 ligand; and
a poly-A region; and
a pharmaceutically acceptable vector to encapsulate the at least one ribonucleic acid (RNA) polynucleotide.

2. The messenger ribonucleic acid (mRNA) vaccine of claim 1, wherein the coding region further comprises a functional sequence encoding a viral replicase.

3. The messenger ribonucleic acid (mRNA) vaccine of claim 2, wherein the functional sequence comprises a self-amplifying functional sequence, so that messenger RNA (mRNA) vaccine is optimized to be a self-amplifying messenger RNA (SAM) vaccine.

4. The messenger ribonucleic acid (mRNA) vaccine of claim 1, wherein the gene of interest comprises a sequence of interest encoding a receptor-binding domain (RBD).

5. The messenger ribonucleic acid (mRNA) vaccine of claim 4, wherein the receptor-binding domain comprises a receptor-binding domain of a spike protein of a severe acute respiratory syndrome coronavirus (SARS-CoV).

6. The messenger ribonucleic acid (mRNA) vaccine of claim 1, wherein the gene of interest comprises a sequence encoding at least one of an S1 subunit and an S2 subunit of a spike protein of a severe acute respiratory syndrome coronavirus 2 (SARS-CoV-2).

7. The messenger ribonucleic acid (mRNA) vaccine of claim 1, wherein the pharmaceutically acceptable vector is selected from the group consisting of lipid nanoparticles (LNP) and polymer nanoparticles (PNP).

8. A method of inducing an antigen-specific immune response in an individual, comprising administering the messenger ribonucleic acid (mRNA) vaccine of claim 1 to the individual of an amount effective to produce an antigen-specific immune response.

9. The method of claim 8, comprising administering the messenger ribonucleic acid (mRNA) vaccine at least once.

10. The method of claim 8, wherein the messenger ribonucleic acid (mRNA) vaccine is administered to the individual by injection selected from the group consisting of a subcutaneous injection and an intramuscular injection.
